(19) United States
(12) Patent Application Publication
(10) Pub. No.: US 2009/0186088 A1
(43) Pub. Date: Jul. 23, 2009

(54) INHALABLE DRUG

(75) Inventors: Hak-Kim Chan, New South Wales (AU); Herbert Clark Chiou, New South Wales (AU); Jian Feng Chen, Beijing (CN); Ting Ting Hu, Beijing (CN); Jimmy Sung Lai Yun, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NanoMaterials Technology Pte Ltd, Singapore (SG)

(21) Appl. No.: 11/908,757

(22) PCT Filed: Mar. 8, 2006
(86) PCT No.: PCT/AU2006/000302

§ 371 Date: Jan. 5, 2009

(30) Foreign Application Priority Data
Mar. 18, 2005 (AU) 2005901355

(51) Int. Cl. A61K 9/14; A61K 31/137

(52) U.S. Cl. 424/484; 514/653

(57) ABSTRACT

A process for preparation of particles of an inhalable drug is described. The process comprises combining a first liquid and a second liquid in a region of high shear, whereby the first liquid and the second liquid interact to form the particles of the drug. One of the first and second liquids comprises the drug or a precursor thereof. In the case where one of the liquids comprises the precursor, the other of the first and second liquids comprises a reagent which reacts with the precursor under high shear conditions to form particles of the drug. In the case where one of the liquids comprises the drug, the other of the first and second liquids comprises a liquid which, when mixed with the liquid containing the drug under high shear, forms particles of the drug.

### TECHNICAL FIELD

The present invention relates to a drug in an inhalable form and to a process for making the drug in an inhalable form,

### BACKGROUND OF THE INVENTION

Control of particle size is an important consideration in production of drugs. This is particularly important when the drug is intended for delivery by inhalation, as particle size has a significant effect on the delivery of particles of the drug to the lungs, and on the degree of irritation to the respiratory tract caused by inhalation of the drug.

Inhalation type drug therapy commonly utilizes a drug together with a suitable carrier. These carriers may be fluorinated materials, such as HFCs (hydrofluorocarbons) and HFAs (hydrofluoroalkanes), which have benefits of low toxicity, inertness, stability and suitable physical properties. However these carriers are well known to be harmful to the environment. Indeed environmental considerations have been responsible for the phasing out of CFCs (chlorofluorocarbons) which have been used in such applications in the past. An alternative to inhalation therapy using a carrier is the use of inhalers which deliver the drug as fine particles without the need for a carrier.

There is therefore a need for a process for producing a drug in the form of suitably fine particles, and for a suitable method for using such a drug in treatment of disease.

### OBJECT OF THE INVENTION

It is an object of the present invention to overcome or substantially ameliorate at least one of the above disadvantages. It is a further object to at least partially satisfy at least one of the above needs.

### SUMMARY OF THE INVENTION

In a first aspect of the reaction there is provided a process for preparation of particles of an inhalable drug comprising combining a first liquid and a second liquid in a region of high shear, whereby the first liquid and the second liquid interact to form the particles of the drug. One of the first and second liquids comprises the drug or a precursor thereof and, in the case where one of the liquids comprises the precursor, the other of the first and second liquids comprises a reagent which reacts with the precursor under high shear conditions to form particles of the drug, and, in the case where one of the liquids comprises the drug, the other of the first and second liquids comprises a liquid which, when mixed under high shear with the liquid containing the drug, forms particles of the drug (i.e. causes particles of the drug to form).

The process may also comprise the step of isolating the particles of the drug. The combining may comprise injecting the first and second liquids into a mixing zone comprising a shear device which imparts high shear to the first and second liquids. The first and second liquids may be injected into the mixing zone directly onto the shear device. The shear device may be rotating in the mixing zone in order to impart high shear to the first and second liquids, and may be rotating at between about 100 and 15000 rpm, or between about 1,000 rpm or less and about 10,000 rpm or more than about 10,000, or more than about 15,000rpm. The first liquid may be miscible with the second liquid. The ratio of the first liquid to the second liquid may be between about 1:200 and about 200:1 on a w/w or v/v basis. The shear device may be substantially cylindrical and may comprise at least one layer of mesh. The shear device may comprise a plurality of overlapping layers of mesh. The mesh may have a mesh size of about 0.05 to about 3 mm or about 0.1 to 0.5 mm or about 0.5 mm or less to 3.0 mm or more. It may have a porosity of greater than about 75%, or greater than about 90%. The shear device may be a rotating packed bed reactor (RPB).

The first and second liquids may be injected into the mixing zone through a plurality of inlets. Each inlet for the first liquid may be located within the mixing region no further than about 15 degree of arc from an inlet for the second liquid. The injection velocity of the first and second liquids may be greater than about 1 m/s, or may be between about 1 and about 120 m/s.

The particles of the drug may be of a size and/or shape suitable for administration by inhalation. The particles may be of a size and/or shape such that, when administered to a patient by inhalation for the treatment of a condition of said patient, the particles are absorbed by the patient at a rate appropriate for treatment of said condition. The particles may be less than about 10 microns in diameter, and may be between about 0.5 and about 10 microns in diameter. The proportion of particles less than about 10 microns in diameter, or between 0.5 and about 10 microns in diameter may be greater than about 50% on a weight or number basis. The sizes of the particles specified herein may be considered to be mean particle sizes. The drug may be an inhalation-type drug. It may be an antibiotic, a β-agonist, a bronchodilator, a steroid, a cyclosporin or some other type of drug. It may be for example tobramycin salmetrol (fluticasone propionate), formotrol, beclomethazone, butazamide or salbutamol sulfate or some other drug.

In one embodiment, the first liquid comprises a solution of the drug in a solvent, and the second liquid comprises a non-solvent for the drug. The non-solvent may be miscible with the solvent, and may be capable of causing the drug to precipitate or form particles when mixed with the solution.

In another embodiment the first liquid comprises a precursor of the drug, optionally dissolved in a first solvent, and the second liquid comprises a reagent, optionally dissolved in a second solvent, whereby the reagent is capable of reacting with the precursor to form the drug. The reagent may be capable of reacting with the precursor to form particles of the drug. The reagent may be capable of reacting with the precursor through an acid-base reaction to form the drug. The ratio of the precursor to the reagent may be between about 5:1 or more and about 1:5 or less on molar basis, and may be between about 3:1 and about 1:3 on a molar basis. The first liquid may be miscible with the second liquid. The first solvent, if present, may be the same as or different to the second solvent, if present, and the two solvents, if present, may be miscible. The drug may be a salt, the precursor may be a free base of the drug and the reagent may be an acid. For example the drug may be salbutamol sulfate, the precursor may be salbutamol and the reagent may be sulfuric acid.

In a further embodiment the process comprises:
providing a mixing zone comprising a high shear device comprising a plurality of overlapping layers of mesh, each of said layers of mesh having a mesh size between about 0.05 mm and about 3 mm;
rotating said high shear device at between about 100 and about 15000 rpm;
providing to the mixing zone a first liquid and a second liquid in a ratio between about 1:200 and about 200:1 on a v/v basis, whereby one of the first and second liquids comprises the drug or a precursor thereof and the other of said first and second liquids, in the case where one of the liquids comprises the precursor, a reagent which reacts with the precursor under high shear conditions to form particles of the drug, and in the case where one of the liquids comprises the drug, the other liquid comprises a liquid which, when mixed with the liquid containing the drug under high shear, forms particles of the drug, thereby forming the particles of the drug having a particle size between about 0.5 and about 10 microns; and
isolating the particles of the drug.

The invention also provides particles of an inhalable drug when prepared by the process of the first aspect of the invention.

In a second aspect of the invention there is provided an inhalable drug in the form of particles having a diameter less than about 10 microns. The particles may have a particle diameter between about 0.5 and about 10 microns. The proportion of particles of the inhalable drug less than about 10 microns in diameter, or between about 0.5 and about 10 microns in diameter may be greater than about 50%, or greater than about 80%, on a weight basis. The particles of the inhalable drug may have a narrow particle size distribution. The proportion of particles of the inhalable drug having a particle size within about 10%, or within about 50%, of the mean particle size may be greater than about 20%, or greater than about 50% on a number or weight basis. The particles may be of a size and/or shape such that, when administered to a patient by inhalation for the treatment of a condition of said patient, the particles are absorbed by the patient at a rate appropriate for treatment of said condition. The particles may be spherical, elongated or acicular or may be an irregular shape or may be some other shape. The particles may be agglomerates. The particles may be prepared by the process of the first aspect of the invention.

In a third aspect of the invention there is provided a method for treating a condition in a patient comprising providing to the patient an inhalable drug in the form of particles, said particles being prepared by the process of the first aspect of the invention. The invention also provides a method for treating a condition in a patient comprising providing to the patient an inhalable drug in the form of particles, said inhalable drug being according to the second aspect of the invention. The inhalable drug may be provided in an inhaler, and the step of providing the drug to the patient may comprise providing the inhaler having particles of the drug therein to the patient. The inhalable drug may be suitable, or indicated, for treatment of the condition, and may be suitable, or indicated, for treatment of the condition by inhalation. The condition may be for example asthma, cancer, an infection such as a lung infection, or it may be some other condition that is treatable by inhalation of a drug.

There is also provided the use of an inhalable drug according to the invention for the treatment of a condition selected from the group consisting of asthma, cancer and an infection. The inhalable drug may be in the form of particles as described above.

In a fourth aspect of the invention there is provided an inhaler for treating a condition in a patient with particles of an inhalable drug therein, said drug being suitable, or indicated, for the condition, and said particles having a particle diameter less than 10 microns. The particles may have a particle diameter between about 0.5 and 10 microns. The particles may be prepared by the process of the first aspect of the invention. The inhalable drug may be an inhalable drug according to the second aspect of the invention. The invention also provides the use of an inhaler according to the fourth aspect for treating the condition in the patient. The use may comprise providing the inhaler to the patient, and allowing the patient to inhale from the inhaler. The patient may be a human patient.

### BRIEF DESCRIPTION OF THE DRAWINGS

A preferred form of the present invention will now be described by way of example with reference to the accompanying drawings wherein:

FIG. 1 is a flow chart of the experiments described in the example;

FIG. 2 is a scheme showing the reaction of salbutamol base with sulfuric acid to produce Salbutamol sulfate;

FIG. 3 shows a photograph of the original precipitate formed in the experiment

FIG. 4 shows a photograph of the precipitate of FIG. 3 after allowing to stand for 2.5 hours;

FIG. 5 is a schematic diagram of the process of beaker synthesis described in the example;

FIG. 6 is a schematic diagram of an RPB (rotating packed bed reactor);

FIG. 7 is a schematic diagram of a MSLI (multi-stage liquid impinger);

FIG. 8 shows a graph of concentration vs. absorption for salbutamol sulfate aqueous is solution at 276 nm;

FIG. 9 shows a graph of reaction time vs. volume medium particle size for the experiment of the example;

FIG. 10 shows a graph of concentration of sulfuric acid vs. volume medium particle size using different concentration of Salbutamol for the experiment of the example;

FIG. 11 shows a graph of reactive temperature vs. volume medium particle size for the experiment of the example;

FIG. 12 shows a graph of stirring speed vs. volume medium particle size for the experiment of the example;

FIG. 13 is a schematic diagram of Salbutamol sulfate suspension samples;

FIG. 14 shows a graph illustrating the effect of sonication on volume medium particle size at different stirring speeds for the experiment of the example;

FIG. 15 shows a graph illustrating the effect of sonication on volume medium particle size using different concentration of sulfuric acid for the experiment of the example;

FIG. 16 is a photograph of a small stirrer head as used in the example;

FIG. 17 is a photograph of a bigger stirrer head as used in the example;

FIG. 18 shows a graph of particle size distributions of salbutamol sulfate precipitated using different reaction times, as measured using a Malvern particle size device;

FIG. 19 shows a graph of volume medium particle size of salbutamol sulfate vs. reaction time, as measured using a Malvern particle size device;

FIG. 20 shows a graph illustrating a relationship between outlet temperature, FPF(total), and FPF(emitted) (FPF is fine particle fraction) for the experiment of the example;

FIG. 21 shows a bar graph illustrating results of Salbutamol sulfate commercial production;

FIG. 22 shows a bar graph illustrating dispersion results with different Salbutamol sulfate spray dry powders;

FIG. 23 shows a bar graph illustrating dispersion results as a function of storage time;

FIG. 24 shows a bar graph illustrating dispersion results as a function of different inhaler device;

FIG. 25 shows a bar graph illustrating the effect of blending with lactose on the dispersion results;

FIG. 26 shows a graph illustrating particle size distribution arising from different reaction temperatures;

FIG. 27 shows a graph illustrating particle size distribution arising from different reaction times;

FIG. 28 shows a bar graph illustrating the effect of different inhaler devices on the dispersion results;

FIG. 29 shows an electron micrograph of the Sept. 7 sample of the example, taken at 20.0 kV 4,000×;

FIG. 30 shows an electron micrograph of the Sept. 7 sample of the example, taken at 20.0 kV 16,000×;

FIG. 31 shows an electron micrograph of the Sept. 8 sample of the example, taken at 20.0 kV 4,000×;

FIG. 32 shows an electron micrograph of the Sept. 8 sample of the example, taken at 20.0 kV 32,000×;

FIG. 33 shows an electron micrograph of the Sept. 13 sample of the example, taken at 5 kV 30,000×;

FIG. 34 shows an electron micrograph of the Sept. 13 sample of the example, taken at 5 kV 50,000×;

FIG. 35 shows an electron micrograph of the Sept. 30 sample of the example, taken at 20.0 kV 4,000×;

FIG. 36 shows an electron micrograph of the Sept. 30 sample of the example, taken at 20.0 kV 16,000×;

FIG. 37 shows electron micrographs showing different shapes of salbutamol sulfate dry powder after spray drying using the non-solvent method: (A) loose powder, and (B) spherical powder;

FIG. 38 shows a bar graph illustrating dispersion results of salbutamol sulfate (Nov. 23 sample) made using the non-solvent method;

FIG. 39 shows a bar graph illustrating dispersion results of salbutamol sulfate (Dec. 13 sample) made using the non-solvent method;

FIG. 40 shows a bar graph illustrating dispersion results of two Salbutamol sulfate dry powder samples described in the example;

FIG. 41 shows a graph illustrating particle size distributions of two kinds of Salbutamol sulfate dry powder samples described in the example;

FIG. 42 shows an electron micrograph of the Sep. 13 sample described in the example; and

FIG. 43 shows an electron micrograph of the Oct. 29 sample described in the example.

### DETAILED DESCRIPTION OF THE PREFERRED EMBODIMENTS

A convenient process for producing particles with well controlled particle size is described in WO02/089970, the contents of which are incorporated herein by cross-reference. WO02/089970 also describes a suitable shear device for performing the process. The shear device may be a rotating packed bed reactor (RPB).

One process for producing particles of a substance with a well controlled particle size comprises combining a solution of the substance in a solvent (a first liquid) with a non-solvent (or anti-solvent) for the substance (a second liquid) in a region of high shear, thereby causing formation of particles of the substance. This general process may be used in the present invention for producing particles of an inhalable drug. Alternatively, one of the first and second liquids may comprise a precursor of the drug and the other of said first and second liquids may comprise a reagent which reacts with the precursor under high shear conditions to form particles of the drug. In the present invention the solvent may be aqueous or non-aqueous, and may comprise water, acetone, ethanol, methanol, isopropanol or some other solvent or some mixture of solvents. The non-solvent may be miscible with the solvent. It may be any non-solvent capable of forming particles of the substance (i.e. of causing the particles to form) when mixed with the first liquid under conditions of high shear, and may comprise, depending on the nature of the solvent and the substance, water, ethanol, acetone, isopropanol or some other liquid or a mixture of liquids. The combining may comprise injecting the first and second liquids into a mixing zone comprising a shear device which imparts high shear to the first and second liquids. The first and second liquids may be injected into the mixing zone directly onto the shear device. The shear device may be rotating in the mixing zone in order to impart high shear to the first and second liquids. The shear device may be rotating at between about 100 and about 15000 rpm, or between about 1,000 rpm and about 10,000 rpm, or between about 1,000 and 5,000, 1,000 and 2,000, 2,000 and 10,000, 5,000 and 10,000 or 2,000 and 5,000 rpm, and may be rotating at about 100, 200, 300, 400, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10000, 11000, 12000, 13000, 14000 or 15000 rpm, or may be rotating faster than 15,000 rpm. The shear rate may depend of the ratio of the first and second liquids. The first liquid may be miscible with the second liquid. The ratio of the first liquid to the second liquid may be between about 1:200 and 200:1, and may be between about 1:200 and 1:1, 1:1 and 200:1, 1:100 and 100:1, 1:20 and 20:1, 1:20 and 10:1, 1:20 and 5:1, 1:20 and 2:1, 1:20 and 1:1, 1:20 and 1:5, 1:20 and 1:10, 1:10 and 20:1, 1:1 and 20:1, 1:2 and 20:1, 1:1 and 20:1, 2:1 and 20:1, 5:1 and 20:1, 10:1 and 20:1, 1:10 and 10:1, 1:5 and 5:1, 1:3 and 3:1, 1:2 and 2:1, 2:3 and 3:2, 1:10 and 1:1, 1:1 and 10:1, or 1:2 and 2:1, and may be about 1:20, 1:15, 1:10, 1:9, 1:8, 1:7, 1:6, 1:5, 1:4, 1:3, 1:2, 2:3, 1:1, 3:2, 2:1, 3:1, 4:1, 5:1, 6:1, 7:1, 8:1, 9:1, 10:1, 15:1 or 20:1, or it maybe some other ratio.

If the formation of the particles is caused by a solvent/non-solvent precipitation (i.e. if one of the first and second liquids comprises the drug), then the ratio of the first and second liquids, and/or the concentration of the drug in one or other thereof, may be such that the combination of the first and second liquids is a sufficiently poor solvent for the drug that the particles are formed. If the formation of the particles is caused by a chemical reaction (i.e. one of the first and second liquids comprises a precursor for the drug and the other comprises a reagent), then the ratio of the first and second liquids, and the concentrations of the precursor and of the reagent, may be such as to cause complete, or near complete, reaction of the precursor to form particles of the drug. Commonly the drug will be relatively expensive compared to the reagent. Consequently it may be advantageous to use a molar excess of the reagent relative to the precursor. The molar excess may be for example 5%, or 10, 15, 20, 25, 30, 40, 50, 60, 70, 80, 90 or 100%, or may be more than 100%. It will be understood that in this context, molar equivalence refers to a ratio between the reagent and the precursor in which there is no excess of either that can not react. Thus for example, if the precursor were to be a diamine and the reagent were to be a monofunctional acid, then molar equivalence would require two moles of reagent per mole of diamine.

The drug may be insoluble, or sparingly soluble, in the combination of the first and second liquids in the ratio in which they are mixed.

The shear device may comprise be substantially cylindrical, or it may be some other shape, for example conical, biconical, ellipsoidal or ovoid. It may comprise at least one layer of mesh. The shear device may comprise a plurality of overlapping layers of mesh. There may be between about 2 and 1000 layers of mesh, or between about 2 and 500, 2 and 100, 2 and 50, 2 and 10, 5 and 1000, 10 and 1000, 50 and 1000, 100 and 1000, 500 and 1000, 5 and 500, 10 and 100, 10 and 50, 5 and 50, 50 and 500 or 50 and 100 layers, and may be about 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900 or 1000 layers. The mesh may have a mesh size of about 0.05 to 3 mm or about 0.1 to 0.5 mm or about 0.5 to 3.0 mm, or about 0.5 or 2.0, 0.5 and 1.0, 1.0 and 3.0, 2.0 and 3.0 or 1.0 and 2.0, and may be about 0.5, 0.6, 0.7, 0.8, 0.9, 1.0, 1.5, 2.0, 2.5 or 3.0 mm. It may have a porosity of greater than about 75%, or greater than about 80, 85, 90, 91, 92, 93, 94, 95, 96, 97, 98 or 99%, and may have a porosity of about 75, 80, 85, 86, 87, 88, 89, 90, 91, 92, 93, 94, 95, 96, 97, 98 or 99%.

The process of combined the liquids in a region of high shear may be achieved by injecting the liquids into a mixing zone comprising a shear means. Preferably the injection is carried out at a high injection velocity of > about 1 m/s more preferably >3 m/s most preferably >5 m/s. It is also preferred that the high shear is provided by rapid rotation of the shear means in the mixing zone leading to shearing of liquids in said mixing zone. The shear may be provided by means of a molecular mixing unit comprising: (a) an outer body defining a mixing zone; (b) a shear means to provide shear to said mixing zone; (c) at least one fluid inlet means for the first liquid ; (d) at least one fluid inlet means for the second liquid; and (e) a fluid outlet means. By the use of such a unit, the precipitation step may be controlled, as the unit allows control of the step of adding the second liquid (for example a coacervation agent) to the first liquid (for example comprising the drug) to control the nucleation and particle growth. The particle size can be controlled in either micron or nano size region by adjusting the rotational speed of the shear means in the mixing zone, by different structural features of the shear means and injecting shear of first and second liquids into the mixing zone at different rates of injection. The outer body of the molecular mixing unit may be made of a number of materials. A suitable material is stainless steel. The body is designed such that it defines a mixing zone. The mixing zone may in theory be any of a number of sizes and the size chosen will depend of the rate of the process to be carried out and the amount of material to be processed. The mixing zone is provided with a shear means located within said mixing zone to impart high shear to the liquids injected into the mixing zone. In principal, the shear means may be any device which imparts high shear on fluid.

In an embodiment of the process of the invention the shear means is rotating in the mixing zone and said first and said second liquids are injected directly onto the rotating shear means. The liquids may be injected simultaneously through separate inlets, and may each be injected via a plurality of inlets. The inlets can be located either around the outside of the mixing zone or may be located so as to deliver the liquids to the centre of the mixing zone. The liquids may be injected through a distributor located in the centre of the mixing region surrounded by the rotating shear means.

The process involves the use of a shear means to impart high shear to the two liquids in the mixing zone. This has the advantage that the two liquids are adequately mixed to form an intimate mixture leading to the formation of a precipitate of the desired size. The shear means preferably comprises a packing with a surface area of about 200-3000 m²/m³, or about 200-1000, 200-500, 500-300, 1000-3000, 500-2000 or 500-1000 m²/m³, or about 200, 300, 400, 500, 600, 700, 800, 900, 10000, 1500, 2000, 2500 or 3000 m²/m³, The packing may be such that it is structured packing or random packing. A packing that may be used is a packing of the wire mesh type packing that may be made from stainless steel, plain metal alloy, titanium metal or plastic or some other suitable material. The shear means may be formed by rolling mesh to form a cylindrical shear means wherein the cylindrical section has sides formed by a plurality of overlapping mesh layers. If it is used, the mesh may have a mesh size of about 0.05 to 3 mm, more preferably about 0.1 to 0.5 mm. The mesh has a preferred mesh porosity of at least about 75%, or at least about 90%, preferably more than 95%. The shear means may be mounted on a shaft in the mixing zone and may rotate in the mixing zone. The shear means may be a cylindrical shape and may define a hollow to accommodate the inlets for the liquids. It will be appreciated, however, that the shape of the container in which the two liquids are combined can also be used to impart shear to the liquids. It is preferred that the shear means rotates in said mixing zone at a sufficient speed to input high shear to said liquids in said zones. The rotation speed is typically of the order of 100 to 15000 rpm, preferably 500 to 12000 rpm, even most preferably 5000 to 8000 rpm. The use of such a strong rotation of the shear means ensures that the two liquids in the mixing zone are subjected to strong shear immediately upon injection. In the process of the invention it is preferred that the liquids are injected into the mixing region by way of a liquid distributor located in the centre of the mixing region in a hollow defined by the rotating shear means. It is preferred that each of the liquids is injected through a plurality of the inlets.

Once the liquids have been mixed and the particles produced the mixture is discharged from the mixing zone and the particles isolated. If the process is carried out as a continuous process which is preferred the addressed liquids are constantly being withdrawn from the mixing zone and the solid isolated. The particles may be isolated by filtration, centrifugation or any other method of isolation of a solid from a liquid. It is preferred that the solid is isolated by filtration.

Whilst not wishing to be bound by theory it is felt that the use of a high shear device in the unit breaks the solution into discrete particles of the two liquids leading to high surface area contact between them leading to the fast precipitation and formation of the desired particles. It is found to be particularly efficient if the two liquids are injected into the mixing zone via separate fluid inlets. Accordingly, preferably the molecular mixing device has at least one fluid inlet for fluid inflow of each of the first and second liquids respectively. Preferably there is a plurality of inlets for each liquid. These liquid inlets may be arranged in a number of ways depending on the structural design of the mixer. The liquid inlets may be located in a distributor which preferably is located in the hollow defined by said shear means. The distributor may define a plurality of inlets for each of the first and second liquids. In an embodiment the liquid inlets alternate on the distributor. In addition, there should be at least one liquid outlet means for draining the molecular mixing device either in a batchwise or continuous fashion.

The shear device may have a gas inlet and a gas outlet for enabling the process to be conducted in a particular atmosphere, for example an anoxic, low oxygen or inert atmosphere. Accordingly, the process may comprise passing a gas, for example nitrogen, helium, argon, carbon dioxide or some other suitable gas, through the region of high shear before and/or during the step of combining the first liquid and the second liquid.

The first and second liquids may be injected into the mixing zone through a plurality of inlets. Each inlet may, independently, be between about 0.5 and 10 mm in diameter, or between about 0.5 and 5, 0.5 and 2, 1 and 10, 1 and 5, 2 and 5, 5 and 10 or 1 and 3 mm in diameter, and may be about 0.5, 1, 1.5, 2, 2.5, 3, 3.5, 4, 4.5, 5, 6, 7, 8, 9 or 10 mm in diameter. Each inlet for the first liquid may be located within the mixing region no further than about 15 degree of arc from an inlet for the second liquid, or less than about 15, 14, 13, 12, 11, 10, 9, 8, 7, 6 or 5 degree of arc, and may be located about 3, 4, 5, 6, 7, 8, 9, 10, 11, 12, 13, 14 or 15 degree of arc from an inlet for the second liquid. The injection velocity of the first and second liquids may be greater than about 1 m/s, or may be greater than about 2, 3, 4, 5, 6, 7, 8, 9, 10, 15, 20, 25, 30, 35, 40, 45, 50, 60, 70, 80, 90, 100, 110 or 120 m/s, or may be between about 1 and 120, 1 and 100, 1 and 50, 1 and 20, 5 and 120, 10 and 120, 50 and 120, 50 and 100, 5 and 50, 5 and 20, 10 and 50, 1 and 10, 1 and 5, 1 and 2, 2 and 10, 5 and 10 or 2 and 5 m/s, and may be about 1, 1.5, 2, 2.5, 3, 3.5, 4, 4.5, 5, 6, 7, 8, 9, 10, 15, 20, 25, 30, 35, 40, 45, 50, 60, 70, 80, 90, 100, 110 or 120 m/s. The injection flow rate may be between about 0.5 and 10 L/min or between about 0.5 and 5, 0.5 and 2, 1 and 10, 1 and 5, 2 and 5, 5 and 10 or 1 and 3 L/min, and may be about 0.5, 1, 1.5, 2, 2.5, 3, 3.5, 4, 4.5, 5, 6, 7, 8, 9 or 10 L/min.

The process may also comprise the step of isolating the particles of the drug. The isolating may comprise filtration, microfiltration, ultrafiltration, centrifugation, ultracentrifugation, settling or a combination of these, or may comprise some other method for separating. Following the isolating, the particles may be dried, for example by vacuum drying, freeze drying, spray drying, flash drying, passing a stream of gas (commonly a dry gas) through or over the particles. The gas may be for example air, nitrogen, carbon dioxide, argon or some other gas or a mixture of gases.

Suitable drugs for the present invention include inhalable drugs. They may be suitable, or indicated, for treatment of asthma, cancer, an infection such as a lung infection, or some other condition. A suitable drug may be one that may be made by reacting a precursor to the drug with a reagent. The precursor and the reagent may be such that they are capable of reacting together to make the drug. They may be capable of reacting together to make the drug under the conditions pertaining in the region of high shear, for example the conditions of temperature and pressure pertaining therein.

The precursor or the drug or both may be in solution. The ratio of the precursor to the reagent may be between about 5:1 or more and about 1:5 or less, and may be between about 3:1 and 1:3 on a molar basis, and may be between about 3:1 and 1: 1, 1:1 and 1:3 or 2:1 and 1:2 on a molar basis, and may be about 3:1, 2.5:1, 2:1, 1.5:1, 1:1, 1:1.5, 1:2, 1:2.5 or 1:3 on a molar basis, or may be some other ratio. The ratio may be such as to encourage or ensure complete conversion of the precursor to the drug. If the precursor or the drug or both are in solution, the concentration of the solution may (independently) be between about 0.1 and 50% w/w or w/v, and may be between about 0.1 and 25, 0.1 and 10, 0.1 and 5, 0.1 and 1, 0.1 and 0.5, 1 and 50, 5 and 50, 10 and 50, 1 and 25 or 1 and 10% w/w or w/v, and may be about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45 or 50% w/w or w/v, or may be some other concentration, depending on the solubility of the solute (i.e. the precursor or the drug) in the solvent. The precursor or the solution of the precursor may be miscible with the precursor or the solution of the precursor. The solvent may be aqueous or non-aqueous, and the solvent for the precursor may be the same as or different to the solvent to the reagent. The solvent for the precursor and for the reagent may, independently, comprise water, ethanol, methanol, isopropanol, acetone or some other solvent or may comprise a mixture of solvents.

The drug may be a salt, the precursor may be a free base of the drug and the reagent may be an acid. The pKas of the reagent and of the precursor may be such that they are capable of reacting to generate the drug. Convenient precursors therefore may be amine functional precursors, whereby the drug is ammonium salt. Suitable drugs may be salts (for example sulfates, hydrochlorides or other salts) of salbutamol, aminophylline, theophylline, orciprenalin, terbutaline, salmetrol, formotrol, beclomethazone, butazamide, mannitol, cyclosporine or tobramycin. The drug may be an inhalable steroid, an inhalable cyclosporine, an inhalable anti-asthma drug, an inhalable bronchodilator, an inhalable antibiotic, an inhalable β-agonist or some other inhalable drug.

In an alternative, the precursor may be a salt of a drug (for example a sulfate, hydrochloride or some other salt) and the reagent may be a base (for example a hydroxide an amine, ammonia), whereby the drug is the free base of the precursor. In this case the drug may be a basic drug, such as an amine functional drug. The reagent may be a stronger base than the drug.

In a further alternative, the drug may be an acidic drug, for example a carboxy functional drug. In this case the precursor may be a salt of the drug (for example a sodium, potassium, ammonium or trialkylammonium salt), and the reagent may be a mineral acid, for example sulfuric acid, hydrochloric acid or phosphoric acid, or an organic acid, for example trifluoroacetic acid.

In a further alternative the drug may be a salt of an acidic precursor, and the reagent may be a base.

In all cases, the pKas of the reagent and precursor should be such that reaction of the reagent and precursor is capable of producing the drug.

The invention also provides an inhalable drug in the form of particles having a diameter (for example a mean particle diameter) less than about 10 microns, or less than about 9, 8, 7, 6, 5, 4, 3, 2 or 1 micron. Particles of greater than about 10 microns commonly are trapped in the mouth and throat when inhaled, and are therefore not effectively delivered to the lungs. Particles below about 0.5 microns appear to have reduced deposition in the lungs, and are therefore not effectively absorbed by a patient. The particles according to the present invention may have a particle diameter between about 0.5 and about 10 microns, and may have a particle diameter of between about 0.5 and 5, 0.5 and 1, 1 and 10, 5 and 10 or 1 and 5 microns, and may have a particle diameter of about 0.5, 1, 1.5, 2, 2.5, 3, 3.5, 4, 4.5, 5, 5.5, 6, 6.5, 7, 7.5, 8, 8.5, 9, 9.5 or 10 microns. The proportion of particles of the inhalable drug less than 10 microns (or less than 9, 8, 7, 6, 5, 4, 3, 2 or 1 microns) in diameter, or between 0.5 and 10 microns in diameter may be greater than about 50%, or greater than about 60, 70, 80, 90 or 95%, on a weight basis, and may be about 50, 55, 60, 65, 70, 75, 80, 85, 90, 95, 96, 97, 98 or 99%. The particles of the inhalable drug may have a narrow particle size distribution. The proportion of particles of the inhalable drug having a particle size within 10% (or 15, 20, 25, 30, 35, 40, 45 or 50%) of the mean particle size may be greater than about 20%, or greater than about 30, 40, 50, 60, 70, 80 or 90% on a weight basis, and may be about 20, 30, 35, 40, 45, 50, 55, 60, 65, 70, 75, 80, 85, 90, 95, 96, 97, 98 or 99% on a number or weight basis. The particles may be any suitable shape. For example they may for example have a shape selected from the group consisting of a sphere, an ellipsoid, a toroid, an ovoid, a modified oval shape, a cone, a truncated cone, a dome, a hemisphere, a cylinder, a round ended cylinder, a capsule shape, a caplet shape, a frustoconical shape, a disc, discoid, a tabular shape, a prismatic shape, an acicular shape and a polyhedron (either regular or irregular) such as a cube, a rectangular prism, a rectangular parallelepiped, a triangular prism, a hexagonal prism, rhomboid or a polyhedron with between 4 and 60 or more faces, or may be some other shape, for example an irregular shape. The particles may be agglomerates, and may be agglomerates of particles having any of the above shapes of particles having some other shape, or of particles having a variety of different shapes.

A drug according to the present invention may be delivered orally to a patient in order to treat a condition in the patient. The drug may be delivered orally, for example using an inhaler. It may be delivered as a dry powder, and may be delivered without the use of a liquid carrier.

### EXAMPLE

### Test Materials

### Reactive Method

Solute: Salbutamol base (NanoMaterials Technology Pte. Ltd., Singapore)
Solvent: Isopropyl alcohol (IPA, AR, BIOLAB, Australia)
Reactant: Sulfuric acid (AR, Phone Poulenc, Australia)
Deionised water

### Non-solvent Method

Solute: Salbutamol sulfate (Inter-Chemical Ltd., China)
Solvent: Deionised water

### Non-solvent: IPA (AR, BIOLAB, Australia)

### Acetone (AR, BIOLAB, Australia)

### Apparatus:

### Beakers (50 ml, 250 ml, 500 ml, 1000 ml)

### Pipettes (5000 μl, Eppendorf, Germany)

### Syringes (Terumo, USA)

Hot-plate Magnetic stirrer (IEC, Australia)

### High Shear Mixer (Silverson, UK)

### Ultrasonic Cleaner (Unisonics, Australia)

Laser diffraction (Malvern Mastersizer, Malvern Instrument, UK)

### Rotating Packed Bed (Bejing University of Chemical Technology, China)

A Büchi Mini Spray Dryer B-191 (Büchi Laboratory-Techniques, Switzerland)
Peristaltic pump (Masterflex C/L, Extech Equipment, Victoria)

### Microscope (Plympus CH40, Japan)

### Scanning Electron Microscope (SEM)

Multi-stage liquid impinger (MSLI, Copley Scientific, Nottingham, UK)

### Oscilloscope (Agilent 54621A)

### Orion Dry-Pump (Orion Machinery, Japan)

### A/E Glass Filter Paper (76 mm, PALL Gelman Sciences, USA)

### H3CR-A8 Timer (Omron, Japan)

Flow Meter (TSI 4000 series, USA)

### Centrifuge (Minispin, Eppendorf, Germany)

Ultraviolet and visible absorption spectrophotometers (UV, HITACHI U-2000, Japan)
Blend machine (Turbula, Switzerland)

### Balance (Mettler Toledo AG245, Australia)

### Flow of Experiment

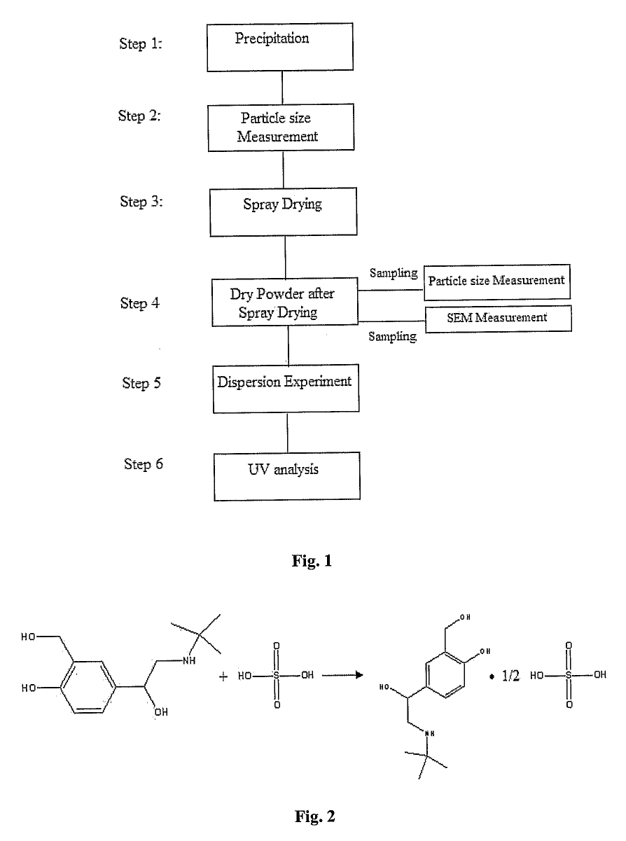

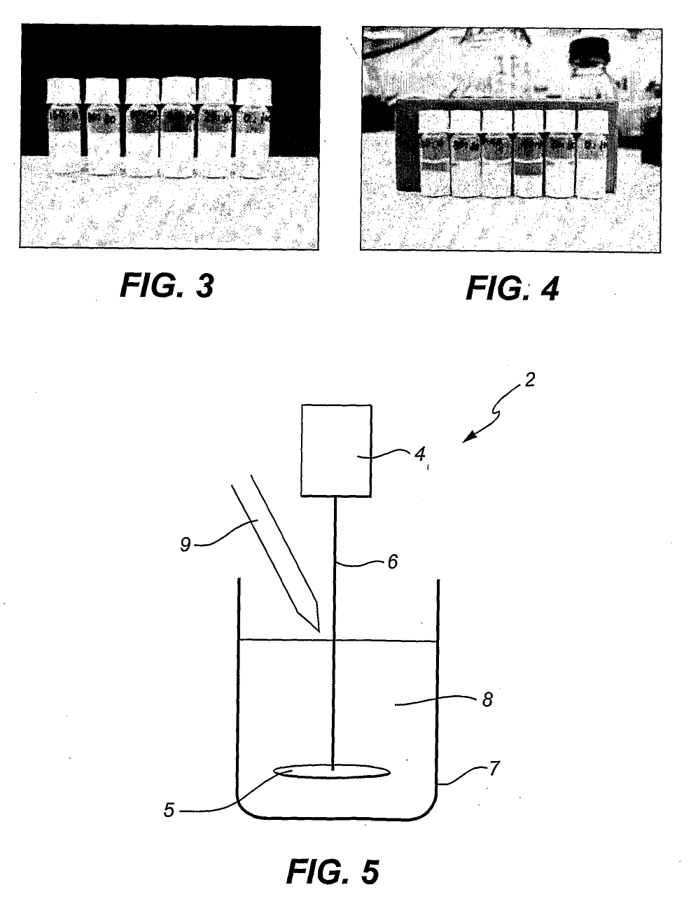

The experiments were designed including 6-step operations (FIG. 1):

### Precipitation

### Reactive Method

There are several route of synthesis for Salbutamol sulfate, but the final step for all is the same, wherein Salbutamol base is reacted with sulfuric acid to produce Salbutamol sulfate (FIG. 2).

### Non-solvent Method

Non-solvent recrystallization is a physical method commonly used to generate nanomaterials. In this method a solute is separated out from the solvent by adding non-solvent (anti-solvent), in which the solute has slight solubility, to change the saturation of the system. Commonly, the lower the solubility of solute in the non-solvent, the more rapidly the solute will separate out. However, if the rate of crystal growth is too fast, the particle size may be big. Therefore, selection of the correct non-solvent is very important in order to form fine particles.

### Choice of Solvent and Non-solvent

### Selection of the Solvent in the Reactive Process

The aim of this experiment was to achieve a suspension containing fine Salbutamol sulfate precipitate. Therefore, a solvent in which Salbutamol base is soluble and in which Salbutamol sulfate is slightly soluble is the correct solvent for the reaction. As is described in British Pharmacopoeia, Salbutamol base is sparingly soluble in water, soluble in alcohol and slightly soluble in ether. Salbutamol sulfate is freely soluble in water, slightly soluble in alcohol and in ether and very slightly soluble in methylene chloride. Alcohol (ethanol) therefore was considered to be the best choice, considering the safety of the possible solvents. The solubility of a solid is a function of the solid particle size according to Equation 1

$$\frac{RT}{M} ln \frac{{S}_{r}}{S} = \frac{2 σ}{rd} \qquad ( Equation 1 )$$

where R is the gas constant, T the absolute temperature, S and Sᵣ the solubility of large solid particles and of particles having small radius r respectively, and M, σ, and d are the molecular weight, surface tension, and density of the solid respectively. It will be appreciated from Eq. 1 that the solubility of a solid will increase as the particle size decreases. Therefore, the solubility of a fine Salbutamol sulfate product (as produced by the process of the present invention) in alcohol will be somewhat enhanced relative to commercial product of larger particle size. Consequently, when one considers the yield of the process, alcohol no longer appears suitable. Isopropyl alcohol (IPA) was therefore chosen to replace alcohol as the solvent in the process, as the solubility of Salbutamol sulfate in IPA is somewhat lower than in alcohol, and the solubility of Salbutamol base in IPA is similar to that in alcohol. Further, IPA is a commonly used organic solvent.

### Selection of the Non-solvent in the Non-solvent Process

About 1 g Salbutamol sulfate (commercial product) was dissolved in 4 ml water to make an approximately saturated aqueous Salbutamol sulfate solution. IPA and acetone were mixed in the following volume ratios: 100:0, 80:20, 60:40,40:60, 20:80, and 0:100. 1 ml of each of the mixture organic solvent was placed as non-solvent into separate 2 ml vials. Then 0.01 ml of the Salbutamol sulfate solution was added to each of the vials containing the non-solvents as rapidly as possible. The vials were then covered and shaken as vigorously as possible by hand. The ratio of solution to non-solvent was 1:100 (0.01 ml:1 ml). The white precipitate began to appear as the solution was added and increased progressively with time. The vials were then allowed to stand, and the precipitate in each vial was observed in order to determine the best volume ratio of IPA to acetone.

Based on the speed of precipitation, IPA to acetone ratios of 80:20, 60:40, and 0:100 appear to be similar and to be better than other three. When the precipitates from these three samples were examined under a microscope, the particle size of the 80:20 sample appeared to have the smallest and best dispersed particles. Consequently, the solvent mixture with IPA:acetone=80:20 v/v was determined to be the non-solvent of choice.

### Beaker Synthesis

A 10 mg/ml solution was prepared by dissolving Salbutamol base in IPA at about 70° and then cooling the solution to room temperature (around 21° C.). 20 ml of this solution was placed in a 50 ml beaker. 0.2 ml of 2 mol/L sulfuric acid (calculated in order to achieve complete reaction of the Salbutamol base and sulfuric acid) was pipetted into the solution using an Eppendorf pipette tip while stirring the solution using an overhead stirrer set at 3000 rpm over a period of 2 minutes to cause precipitate formation (FIG. 5). In FIG. 5, apparatus 2 comprises overhead stirrer 4 attached to blade 5 by shaft 6 and immersed in 250 ml beaker 7. Solution 8 in beaker 7 is the solution of Salbutamol base in IPA. Sulfuric acid may be added using Eppendorf pipette 9.

### The Process of RPB (Rotating Packed Bed Reactor) Synthesis

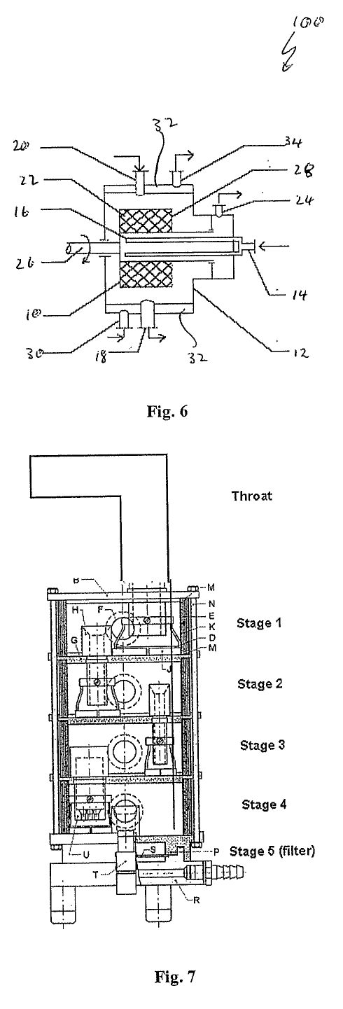

With reference to FIG. 6, RPB 100 comprises packed rotator 10, which is installed inside stationary shell 12 and rotates at a speed of several hundreds to thousands of revolutions per minute. Liquid is introduced into the eye space of rotator 10 from the liquid inlet 14 and sprayed onto the inside edge of rotator 10 through the slotted liquid distributor 16. The liquid in the bed flows in the radial direction from the inside edge to the outside edge under centrifugal force, finally collects and leaves RPB 100 via liquid outlet 18. Gas is introduced through gas inlet 20 and flows inward countercurrently to the liquid in packing 22 of rotator 10 and finally flows out from gas outlet 24. Shaft 26 is connected to rotator 10 in order to provide rotational energy from a motor (not shown). Seal 28 is provided to prevent leakage of the liquid in the bed. Water is injected, if required, through water inlet 30, in order to provided cooling or heating to reactor 100 by means of water jacket 32, and exits jacket 32 through water outlet 34.

The basic principle of the RPB is the creation of a high-gravity environment via the action of centrifugal force, whereby mass transfer and micro-mixing may be intensified. Liquid passing through the packing is spread or split into micro- or nano-droplets, threads or thin films under the high gravity environment in the RPB, which may be of the order of several hundred or even several thousand times greater than the gravitational acceleration of the earth. The rate of mass transfer between gas and liquid or liquid and liquid in an RPB may therefore be 1 to 3 orders of magnitude larger than that in a conventional packed bed, leading to a dramatic reduction of the reaction time required.

### Mode of the RPB Operation

Step 1: Switch on all the power.
Step 2: Add 100 ml Salbutamol base/IPA solution via the top inlet into RPB after ensuring the whole system is clean. Then plug with a rubber stopper tightly. Turn on the cooling/heating system if necessary.
Step 3: Connect a recycling tube fitted to a peristaltic pump for recycling the output from the RPB back through the RPB. (It should be noted that the RPB used in this example has an inlet for admitting the Salbutamol base/IPA solution and an injection port for injecting the acid. There is a recycling port to which the recycling tube may be connected, so that the output from the RPB may be recycled.)
Step 4: Turn on the peristaltic pump at a speed of 2000 ml/min, and then turn on the RPB to the maximum speed around 3000 rpm.
Step 5: Feed sulfuric acid into RPB with a syringe pump or syringe after the RPB has reached its set speed. Since the volume of acid is generally very small, it may usually be added immediately, i.e. within several seconds.
Step 6: The liquid is recycled through the reactor for the required reaction time. After the reaction time has elapsed, the RPB is turned off, and then the pump is turned off. The suspension is then collected in a container of suitable size, for example a 250 ml beaker. This is achieved by disconnecting the outlet tube and allowing gravity to drain the suspension from the RPB.

### Particle Size Measurement

The volume median particle size D(V,0.5) and particle size distribution of the samples obtained immediately after precipitation were measured by laser diffraction (Malvern Mastersizer) using a 2.4 mm active beam length, having calibrated and warmed up the laser for half an hour. The following settings were used:

Set-up range: 300RF (0.05-900 μm)

Sample unit: MS17

Instrument port: 2

Analysis model: Polydisperse

Data channels: low-0, high-0

Presentation: 3_SALB_{—}3 (η_{Salbutamol sulfate}=1.5530, imagine refractive index=0.1000, η_{IPA}=1.378).

For measuring the particle size as a precipitate, the small volume sample dispersion unit was filled with 70 to 100 mL of blank solution—isopropyl alcohol (propan-2-ol, IPA) and the dispersion unit controller was set to mix at 2000 rpm to first calibrate by measuring the background. The suspension was then introduced into the dispersion unit until the obscuration was between about 10 and 30%. This enabled the measurement of the size of the dry particles. Particle size distributions were expressed in terms of volume (or mass) median diameter (D(v, 0.5)), such that the size quoted is at 50% of the entire volume distribution. The distribution width, or span is calculated according to Equation 2, where D(v,0.9) and D(v,0.1) are the respective diameters at 90 and 10% cumulative volume.

$$Span = \frac{D ( v , 0.9 ) - D ( v , 0.1 )}{D ( v , 0.5 )} \qquad ( Equation 2 )$$

### Spray Drying

A Büchi Mini Spray Dryer B-191 with 7 mm nozzle was chosen as a method of recovering powder though the suspension. It was first heated to a preset inlet temperature of 150° C., atomizer at 800 L/hr and 100% aspiration for at least 30 minutes after checking that a clean filter bag was assembled and the air dehumidifier (CompAir SAM35) was switched on. The Salbutamol sulfate suspension was fed via tubing through a peristaltic pump at 5 ml/min into the atomizer nozzle of the spray dryer. The suspension was then atomized, and the IPA evaporated to leave the dried Salbutamol sulfate particles. These particles were then collected using a vacuum pump in the small high efficiency cyclone and collection vessel. The nozzle of the spray dryer was constantly checked and cleaned with distilled water to prevent blockage by the dry powder. The powder was stored in a desiccator over silica gel until used, to avoid aggregation and moisture adsorption during storage.

### Scanning Electron Microscope (SEM)

An SEM was used to examine the morphology of the particles produced, and to confirm the physical size as a means of checking the reliability of the Malvern. Samples were mounted onto copper plates and platinum coated prior to analysis.

### Dispersion Experiments

The measurement of particle sizes in vitro allows a simplistic estimation of the aerodynamic particle size distribution. This process utilizes cascade or impactor devices such as the multi-stage liquid impinger (MSLI) (FIG. 7) to provide a breakdown of the particle size distribution through separation and collection of powder of different Dₐ.

The MSLI consists of 5 stages which have been calibrated at 60 L/min to collect powder of a certain Dₐ, known as the cut-off Dₐ:

Stage 1: 13.0 μm

Stage 2: 6.8 μm

Stage 3: 3.1 μm

Stage 4: 1.7 μm

a greater Dₐ are collected on the higher stages. To convert the calibrated Dₐ (D₁) for each stage at different flow rates (Q₂), Equation 3 was used to find D₂, the new cut-off aerodynamic diameter.

$${D}_{2} = {D}_{1} \sqrt{( \frac{{Q}_{1}}{{Q}_{2}} )} \qquad ( Equation 3 )$$

At flow rates of 60-100 L/min, the particles which have a Dₐ of less than 5 μm are expected to be able to surpass oropharangeal deposition and the filter of the MSLI from equation 3. From the results of the MSLI, the FPF (fine particle fraction) can be calculated, and expressed as two different forms:

$$\begin{matrix}{FPF}_{total} = \frac{powder mass on stage 3 + stage 4 + filter}{\begin{matrix}total mass of powder \\ collected from all components\end{matrix}} & ( Equation 4 ) \\ {FPF}_{emitted} = \frac{powder mass on stage 3 + stage 4 + filter}{\begin{matrix}total mass of powder \\ collected \text{-} [ amount in capsules + device ] \end{matrix}} & ( Equation 5 )\end{matrix}$$

The FPFₜₒₜₐₗ value represents the amount of powder which is of an optimal size for pulmonary deposition in relation to the total amount of powder which would theoretically be inhaled by the patient. This takes into account the powder which does not disperse and remains in the capsules and device. This represents the most practical predictor of the in vivo situation, and is therefore the more quoted result of the two. Nevertheless, the FPF_{emitted} is calculated as another descriptor of aerosol delivery, which does not take into account the powder which remains undispersed.

The recovery was also calculated for each dispersion which represents the mass of powder which was accounted for compared with the mass which was loaded into the capsules (Equation 6).

$$Recovery = \frac{\begin{matrix}sum of mass of powder \\ recovered after dispersion\end{matrix}}{\begin{matrix}total mass of powder \\ measured into capsules\end{matrix}} \qquad ( Equation 6 )$$

### Mode of Dispersion

The equipment used to perform the dispersion experiments was the MSLI, which has been described earlier. The usual mode of operation for Salbutamol sulfate is as following:
Step 1: 3 capsules (vegetable capsule) filled with 10.00±0.50 mg Salbutamol sulfate powder were accurately weighed by balance.
Step 2: A known volume (20 ml) of pure water was put into each of the four stages via 5000 μl Eppendorf pipette. The MSLI was shaken to permit the bottom of each stage to completely wet, especially the sintered glass impaction plate.
Step 3: A piece of Type A/E Glass (76 mm) filter paper was placed at stage 5.
Step 4: The MSLI was assembled with an Orion Dry-Pump, which generates inspiratory flow, and an H3CR-A8 timer, which controls the air flow time.
Step 5: The air flow pattern was checked by oscilloscope to ensure there was no upward spike in the oscilloscope pattern, which would signify a blockage restricting flow through the impinger.
Step 6: The air flow rate was adjusted to 60 L/min using the flow meter through inhaler which contained an empty capsule and MSLI with filter paper inside.
Step 7: The capsules filled in Salbutamol sulfate were loaded and dispersed for 4 seconds each.
Step 8: After dispersion, the 4 stages were swirled thoroughly to dissolve the impacted powder from bottom, top, and side walls,
Step 9: The throat, the device including the adaptor, and the capsules were individually rinsed with 20 ml pure water.
Step 10: The filter paper was also rinsed with 20 ml pure water, and the rinsing water placed in a centrifuge and spun at 13.4×1000 pm for 20 minutes. The supernatant was then collected.
Step 11: All rinsings were collected into separately labeled glass vials and analyzed by UV immediately to minimize the effect of evaporation, which would produce an overestimation of the concentration collected.

### Ultraviolet and Visible Absorption Spectrophotometry(UV)

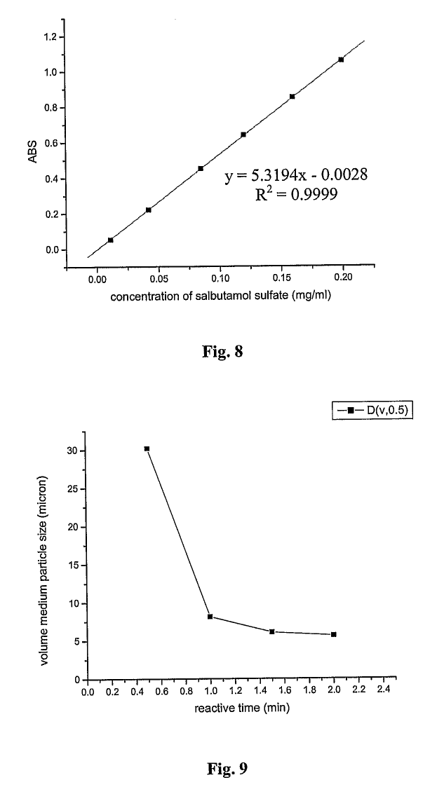

Aqueous salbutamol sulfate standards of known concentrations were prepared and the absorbance of each standard were measured at 276 nm with pure water as background. A standard calibration curve for Salbutamol sulfate aqueous solutions (FIG. 8) was derived from the absorbance and concentration data.

### Results and Discussion

Several reaction conditions were varied in order to investigate which factors of the reaction process influenced the particle size of the product. The five factors investigated were: 1) the stirring speed of the overhead stirrer; 2) reaction temperature; 3) reaction time; 4) concentration of Salbutamol base/IPA solution; and 5) concentration of sulfuric acid.

### Effect of Reaction Time

In this experiment, the reaction time was varied, and the concentration of Salbutamol sulfate/IPA solution, the concentration of sulfuric acid and the stirring speed were maintained constant, as shown in Table 1.

TABLE 1
Effect of varying reaction time
Constant conditions

| | | | |
|---|---|---|---|
| 1 | C_{salbutamol.} | 10 | mg/ml(IPA) |
| 2 | C_{H2SO4} | 2 | mol/L |
| 3 | Stirring speed | 5000 | rpm |
| 4 | Reaction temperature | 20° | C. |

Varied condition

| | | | |
|---|---|---|---|
| reaction | 1 | 0.5 | min |
| time | 2 | 1 | min |
| | 3 | 1.5 | min |
| | 4 | 2 | min |

As shown in FIG. 9, reaction time has a marked effect on D(v,0.5) especially at the short reaction times. The volume medium particle size decreased rapidly from approximate 30 μm to 8 μm during the first half minute, and then reduced smoothly to about 5 μm. Consequently, it appears that long reaction times favor fine particles. In order to save time, 2 minutes was used in subsequent experiments.

### Effect of Concentrations of Sulfuric Acid and Salbutamol Base/IPA Solution

The concentration of sulfuric acid and the concentration of Salbutamol base/IPA solutions were considered likely to affect the results of experiment. The following experiments varied the concentration of sulfuric acid from 0.5 mol/L to 5 mol/L, reacting with 10 mg/ml and 15 mg/ml Salbutamol base/IPA solution separately. Reaction time was kept constant at 2 minutes, reactive temperature was 20° C. and the stir speed was 5000 rpm (Table 2).

TABLE 2
The effect of concentration of sulfuric acid
and Salbutamol base/IPA solution conditions
Constant conditions

| | | | |
|---|---|---|---|
| 1 | Stir speed | 5000 | rpm |
| 2 | Time | 2 | min |
| 3 | temperature | 20° | C. |

Varied conditions

| | | | |
|---|---|---|---|
| 1 | C_{salbutamol.} | 1 | 10 mg/ml |
| | | 2 | 15 mg/ml |
| 2 | C_{H2SO4} | 1 | 0.5M |
| | | 2 | 1M |
| | | 3 | 1.5M |
| | | 4 | 2M |
| | | 5 | 3M |
| | | 6 | 4M |
| | | 7 | 5M |

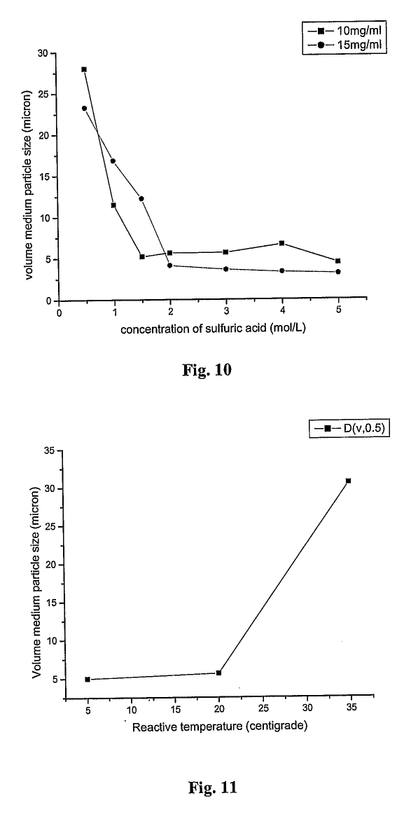

Higher concentrations of sulfuric acid and Salbutamol base both appear to favour production of fine particles (FIG. 10). The inventors consider that this may be due to higher concentrations giving rise to higher super-saturation of the reaction system. In view of the corrosive nature of more concentrated acid, and the consequent effect on the equipment, 2 mol/L was considered to be an appropriate concentration for the reaction. Further, when the concentration of Salbutamol base/IPA solution was increased, the fluidity of the Salbutamol sulfate suspension is reduced. This had an adverse effect on the subsequent step of spray drying to isolate the dry powder. Accordingly, 10 mg/ml Salbutamol base/IPA solution was considered to be better than 15 mg/ml.

### Effect of Reaction Temperature

Using the constant reaction conditions shown in Table 3, only reaction temperature was varied, from 5° C. to 35° C.

TABLE 3
The effect of reactive temperature conditions
Constant conditions

| | | | |
|---|---|---|---|
| 1 | C_{salbutamol.} | 10 | mg/ml(IPA) |
| 2 | C_{H2SO4} | 2 | mol/L |
| 3 | Stirring speed | 5000 | rpm |
| 4 | Time | 2 | min |

Varied condition

| | | | |
|---|---|---|---|
| reaction | 1 | 5° | C. (ice and water) |
| temperature | 2 | 20° | C. (room temperature) |
| | 3 | 35° | C. (hot water) |

FIG. 11 illustrates the influence of the reaction temperature on D(v,0.5). The D(v,0.5) of Salbutamol sulfate was sharply increased when the temperature was above room temperature (around 20° C.), but only slightly increased when the temperature was increased from the low temperature to room temperature. A possible explanation is that the solubility of Salbutamol sulfate in IPA is lower at low temperature than at high temperature. Therefore, a high degree of super-saturated solution may readily be achieved at low temperature, this being one of the key factors required to produce fine particles. Although the particle size was somewhat smaller at 5° C. reaction temperature than at 20° C., the inconvenience of reducing the reaction temperature was considered to outweigh the slight benefit obtained, and reactions were consequently frequently performed at room temperature.

### Effect of Stirring Speed

To investigate the effect of stirring speed, stir speed was the only varied parameter, and the other four parameters were kept constant, as shown in Table 4.

TABLE 4
The effect of stir speed of overhead stirrer
Constant conditions

| | | | |
|---|---|---|---|
| 1 | C_{salbutamol.} | 10 | mg/ml(IPA) |
| 2 | C_{H2SO4} | 2 | mol/L |
| 3 | Temperature | 5° | C. |
| 4 | Time | 2 | min |

Varied condition

| | | |
|---|---|---|
| Stirring | 1 | 1000 rpm |
| speed | 2 | 2000 rpm |
| | 3 | 3000 rpm |
| | 4 | 4000 rpm |
| | 5 | 5000 rpm |

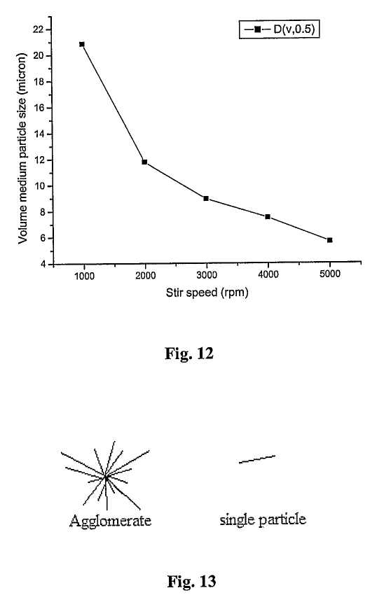

The volume medium particle size (D(v,0.5)) decreased with increasing stirring speed of the overhead stirrer (FIG. 12). This was likely due to faster speed leading to better micro-mixing (i.e. mixing on the molecular scale) to control nucleation and crystallization of the particles. Consequently, the maximum speed which the machine could reach was chosen to achieve finer particles.

### Effect of Sonication

When a small sample was withdrawn from the Salbutamol sulfate suspension after reaction to make a microscope sample, it was observed that the particles were agglomerated rather than separated (FIG. 13).

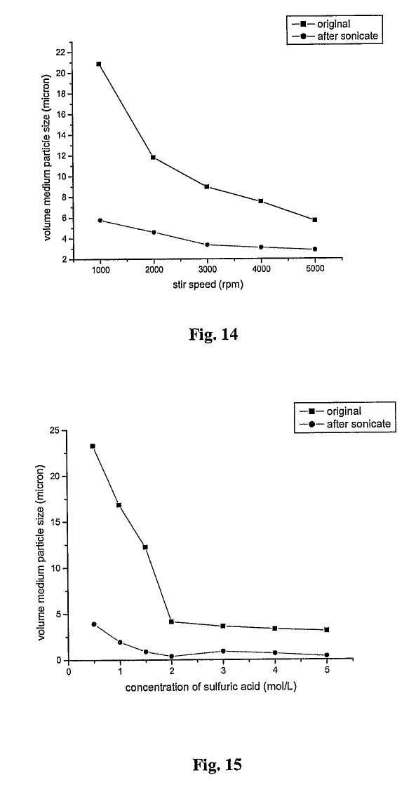

It was therefore considered that the volume medium particle size measured by Malvern may be that of agglomerates rather than of individual particles. In order to test this, the samples were put into ultrasonic bath to sonicate for 30 minutes before measured the particles size. Ice was added to the bath to control the temperature of water in the bath during the sonication process, in order to avoid the influence of temperature on particle size of Salbutamol sulfate. The results are shown in FIG. 14 and FIG. 15. After sonication, the D(v,0.5) was reduced for each sample, and the values after sonication are more consistent with the single particle sizes measured under the microscope. Thus it appears that most of individual particles were separated from the agglomerates by sonication. It is thus clear that agglomerates do exist and the drying technique (spray drying) does not completely break down the agglomerates. Consequently the size of the agglomerates is more relevant than the size of the individual particles that compose the agglomerate until a new technique becomes available which can break agglomerates in the process.

### Experiment Factorial Design

Optimal reactive conditions, such as concentration, temperature were selected from the above experiments. A partial 3⁴ factorial design was employed to investigate which factors concerned with the reaction process influenced the particle size to the greatest extent. The four factors investigated were: concentration of Salbutamol base/IPA solution, concentration of sulfuric acid, stirring speed, and reaction time. The levels investigated for each factor are given in Table 5. Reaction temperature was kept constant at room temperature.

TABLE 5
Factors and the levels used in the experiment factorial design

| | | | Levels | | |
|---|---|---|---|---|---|
| notation | Factor | | 1 | 2 | 3 |
| A | C_{salbutamol.} | mg/ml(IPA) | 10 | 15 | 20 |
| B | C_{H2SO4} | mol/L | 1 | 2 | 3 |
| C | stir speed | rpm | 3000 | 5000 | 8000 |
| D | reaction time | min | 0.5 | 1 | 2 |

TABLE 6
Results from the factorial design analysis, using volume medium particle size data

| | | | | | | D(v, 0.5) |
|---|---|---|---|---|---|---|
| No. | | A | B | C | D | (micron) |
| (1) | | 1 | 1 | 1 | 1 | 15.41 |
| (2) | | 1 | 2 | 2 | 2 | 5.42 |
| (3) | | 1 | 3 | 3 | 3 | 2.81 |
| (4) | | 2 | 1 | 2 | 3 | 5.71 |
| (5) | | 2 | 2 | 3 | 1 | 4.13 |
| (6) | | 2 | 3 | 1 | 2 | 5.30 |
| (7) | | 3 | 1 | 3 | 2 | 7.09 |
| (8) | | 3 | 2 | 1 | 3 | 9.14 |
| (9) | | 3 | 3 | 2 | 1 | 20.24 |
| k₁ⱼ | | 23.64 | 28.21 | 29.85 | 39.78 | 75.25 |
| k₂ⱼ | | 15.14 | 18.69 | 31.37 | 17.81 | |
| k₃ⱼ | | 36.47 | 28.35 | 14.03 | 17.66 | |
| k̅₁ⱼ | (kᵢⱼ/3) | 7.88 | 9.4 | 9.95 | 13.26 | |
| k̅_{2J} | | 5.05 | 6.23 | 10.46 | 5.94 | |
| k̅₃ⱼ | | 12.16 | 9.45 | 4.68 | 5.89 | |

| | | | | | |
|---|---|---|---|---|---|
| Rⱼ | 7.11 | 3.22 | 5.78 | 7.37 | D > A > C > B |
| (max k̅ᵢⱼ-min k̅ᵢⱼ) | | | | | |
| | A2 < A1 < A3 | B2 < B1 < B3 | C3 < C1 < C2 | D3 < D2 < D1 | |
| min | 5.05 | 6.23 | 4.68 | 5.89 | |
| OPTIMAL | A2 | B2 | C3 | D3 | |

Rⱼ is the value which reveals which factor affects the results to the greatest degree: the larger the value, the more sensitive, the greater the sensitivity to that factor. As calculated in Table 6, the values for Rⱼ are in the order D>A>C>B, highlighting that reaction time is the most important of the four factors. Concentration of sulfuric acid from 1 mol/L to 3 mol/L produced the least effect on D(v,0.5).

The minimum value of k̅ⱼ is the value which reveals which level of each factor is the optimal. Table 6 shows that A2, B2, C3, and D3 are the best conditions. Referring to Table 5, these are: C_{salbutamol} 15 mg/ml, C_{H2SO4} 2 mol/L, stirring speed 8000 rpm, and reaction time 2 minutes. These conditions are consistent with the experiments described earlier.

### Synthesis Scale Up

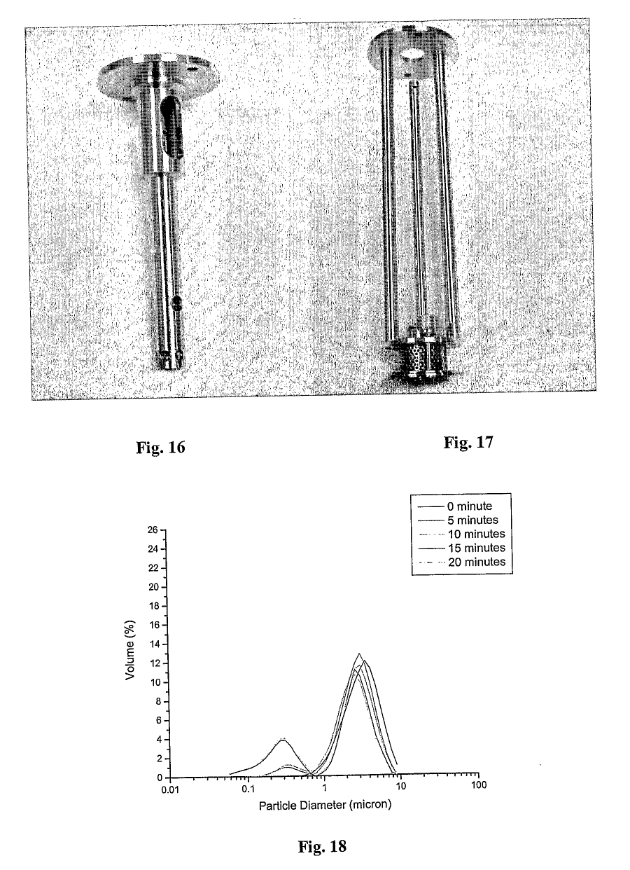

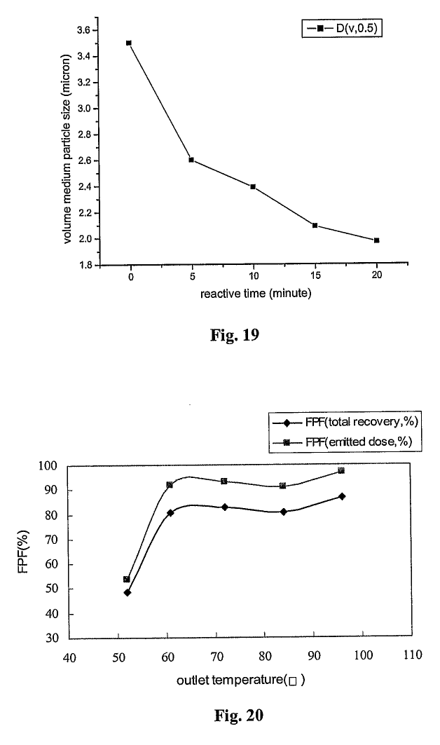

Beaker synthesis of Salbutamol sulfate was scaled up in order to gain enough powder through spray drying for further experiments.
1) 100 ml of Salbutamol base/IPA solution in a 250 ml beaker replaced the 20 ml in a 50 ml beaker as described earlier.
2) A larger stirrer head was used. Comparing the two stirrer heads, not only the size is larger, but also the type is different (FIG. 16, 17). The larger head is more efficient for achieving the fine particles.
3) From the factorial design experiment, it was found that the reaction time was the most important factor in determining the particle size. Thus the reaction time was extended from 2 minutes to 20 minutes, measuring the particle size every 5 minutes (FIG. 18, 19). As the reaction progressed, the particle size distribution curve became bimodal, i.e. a small peak appeared after about 5 minutes, and become progressively larger. Accordingly, D(v,0.5) decreased from 3.50 μm to 1.97 μm after 20 minutes reaction. This confirmed that long reaction time favors smaller particle size.
   In conclusion, the reaction conditions after scale up were as shown in Table 7.

TABLE 7
The scale up reactive conditions

| 1 | Salbutamol base/IPA solution | 10 | mg/ml(IPA), |
|---|---|---|---|
| | | 100 | ml |
| 2 | Sulfuric acid | 2 | mol/L, |
| | | 1 | ml |
| 3 | Reaction Temperature | 20° C. | |
| 4 | Reaction Time | 20 | min |
| 5 | Stirring speed | 8000 | rpm |

### Conditions of Spray Drying

Two values, volume medium particle size (measured by Malvern) and FPF (fine particle fraction) value of the dispersion experiment, were used to indicate the quality of dry powder after spray drying.

The principle of spray drying is that atomizing the suspension through a nozzle, in combination with hot air, causes evaporation of the solvent (in the present example IPA) due to the high temperature. Consequently the solids which are suspended in the suspension system are left as a dry powder which may be collected. Therefore, the inlet and outlet temperature should be high enough to ensure complete evaporation of the solvent. The outlet temperature of spray drying is related to the inlet temperature and can not be preset or controlled independently. The boiling point of IPA is 73° C., so both of the inlet and outlet temperature should be above this value. FIG. 20 illustrates the relationships between outlet temperature, FPF(total) and FPF(emitted).

As shown in FIG. 20, when the outlet temperature is under 60° C., the powder may not dry completely, and thus both FPF values are low. Experiments have been conducted which show that if the inlet temperature is 150° C., corresponding outlet temperature may reach approximate 100° C. and that this is the maximum value that should be used in order to obtain high quality dry powder.

### Dispersion Results

### Commercial Product

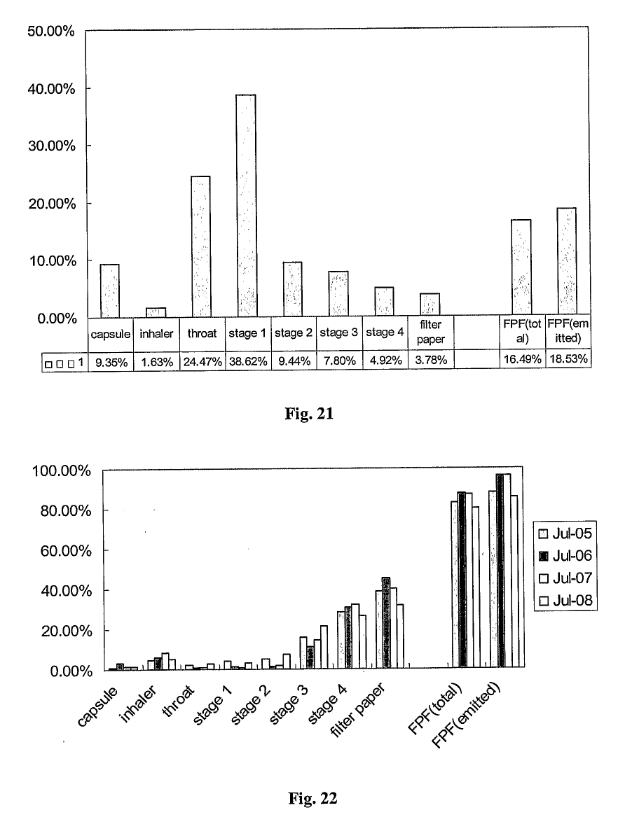

A commercial Salbutamol sulfate product was provided by NanoMaterial Technology Pte Ltd. (Singapore). The dispersion experiment was conducted twice as described earlier. Results of these experiments are shown in FIG. 21 and FIG. 22. D(v,0.5), as measured by the Malvern Mastersizer, was 15.12 μm, which was larger than the cut-off of stage 1 of the MSLI. Neither of the FPF values was above 20%, which means that less than 20% of the current Salbutamol sulfate commercial product is capable of depositing in deep lung area. This result is very unsatisfactory.

Comparison with Different Salbutamol Sulfate Spray Dried Powders Synthesized in Beaker Under Same Reactive Conditions and Spray Drying Conditions

TABLE 8
Reaction conditions and spray drying conditions of Salbutamol sulfate synthesized in a beaker

| | | | |
|---|---|---|---|
| July 5 | July 6 | July 7 | July 8 |

Reaction conditions

| 1. Salbutamol IPA solution: | C = 10 | mg/ml, | V = 100 | ml, | m = 1000 | mg, | n = 4.179 | mmol |
|---|---|---|---|---|---|---|---|---|
| 2. sulfuric acid | C = 2 | mol/L, | V = 1.0 | ml, | n = 2 | mol | | |
| 3. stirring speed | 8000 | rpm | | | | | | |
| 4. reaction temperature | Room temperature | | | | | | | |
| 5. reaction time | 20 | mins. | | | | | | |

| | |
|---|---|
| 6. sonication | no |

Spray drying conditions

| 1. inlet preset | 150° C. | | | | |
|---|---|---|---|---|---|
| 2. outlet temperature | 89° C. | | 92° C. | 90° C. | 86° C. |
| 3. aspirator | 100% | | | | |
| 4. air flow rate | 800 | L/hr | | | |
| 5. feed flow rate | 5 | ml/min | | | |

| | |
|---|---|
| 6. sonication | no |

Results

| | | | | |
|---|---|---|---|---|
| 1. yield ratio | 60.2% | 52.3% | 67.4% | 58.4% |
| 2. FPF (base on total) | 82.90% | 87.23% | 86.79% | 79.95% |
| 3. FPF (base on emitted | 87.75% | 96.20% | 96.18% | 85.47% |
| dose) | | | | |

FIG. 22 illustrates that the method used to generate Salbutamol sulfate dry powder has very good repeatability with regard to dispersion results. Compared with the commercial product, both FPF values of the powder obtained by spray drying show marked improvement, achieving values rarely obtained hitherto. The main cause for this result is that D(v,0.5) of the spray dried powder is under 2 μm, which is smaller than the cut-off of stage 3 (3.1 μm). Therefore most of powder can pass through stages 3 and 4 and collect on the filter paper in Stage 5. The recovery of the powder was more than 90%. It should be noted that while D(v, 0.5) is the physical size of a particle in suspension, aerodynamically a particle may behave like a smaller particle.

### Effect of Storage

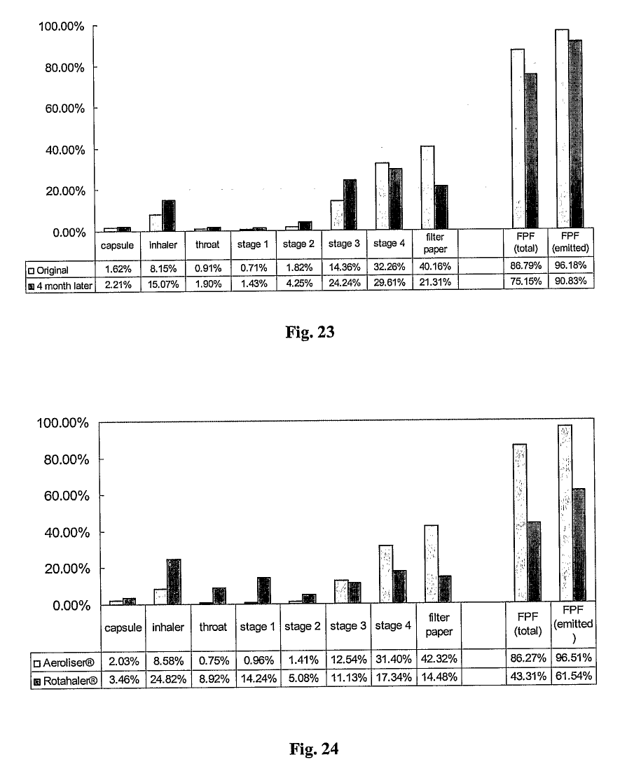

The powder, after spray drying, was collected in glass vials and stored in desiccators in order to minimize the influence of humidity on the dry powder. Comparison of the dispersion results measured immediately and measured 4 months later for the same sample (FIG. 23) show that after 4 months storage the powder depositing distribution changed somewhat. After 4 months, more powder deposited in stage 3 while less stuck on the filter paper in stage 5, relative to the results measured before storage. Thus the proportion of particles under 1.7 μm was decreased while the proportion over 3.1 μm increased. The particles became a little larger on storage for 4 months. Although the FPF(total) and FPF(emitted) reduced by about 11% and 6% respectively, the values are still high compared with commercial products, and powder still can be considered to be high-performance for inhalation.

### Effect of Different Inhalation Device

Rotahaler® is a low-efficiency inhalation device. Due to the excellent performance of the powder, as demonstrated earlier, it was considered that the powder might be of a sufficient quality to perform reasonably well, even in a low-efficiency inhalation device such as the Rotahaler® compared to the higher efficiency Aeroliser®, which was used in the earlier experiments. The powder was dispersed, using identical conditions (60 L/min air flow rate, 4 seconds each time, and same amount of the Salbutamol sulfate), but using two different inhaler devices (FIG. 24). As expected, the performance of Rotahaler® was not as good as Aeroliser®. Approximate 20% and 30% reduction in FPF was observed for FPF(total) and FPF(emitted) respectively. However, these values are still about twice to three times those of the commercial product using Aeroliser®. Thus it may be concluded that fine particles have high-performance even using a low-efficiency inhalation device.

Effect of Blending with Lactose

Lactose is a commonly used carrier for dry powder inhalation. Salbutamol sulfate spray dried powder was blended with lactose (commercial product) at a ratio of 10:90. The experimental details were as follows:

Step 1: Put 100 mg lactose in a small vial. Then add 100 mg Salbutamol sulfate spray dried powder. Use a blending machine to blend them for 1 minute.

Step 2: Add 200 mg lactose into the blended powder, then blend together for 1 minute.

Step 3: Add 400 mg lactose and blend as above.

Step 4: Add 200 mg lactose and blend as above. 1000 mg blended powder was obtained, which contained 100 mg Salbutamol sulfate spray dried powder and 900 mg lactose.

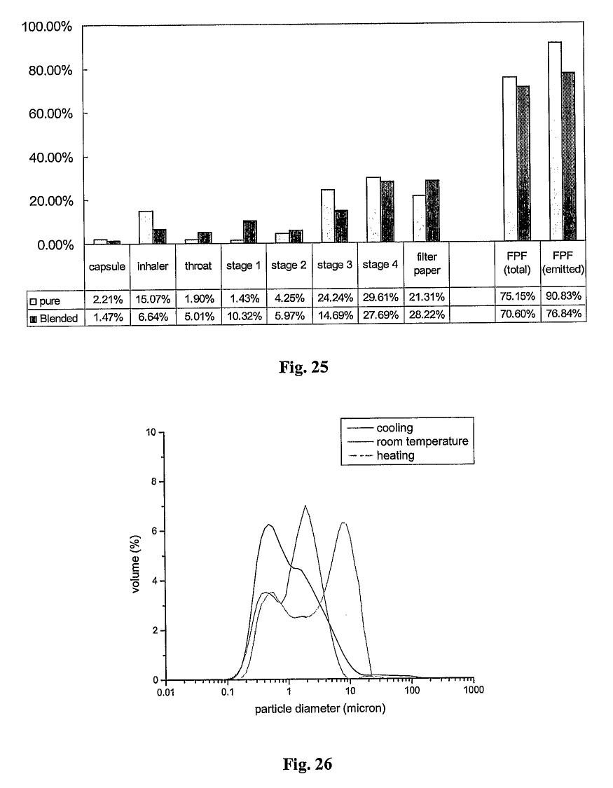

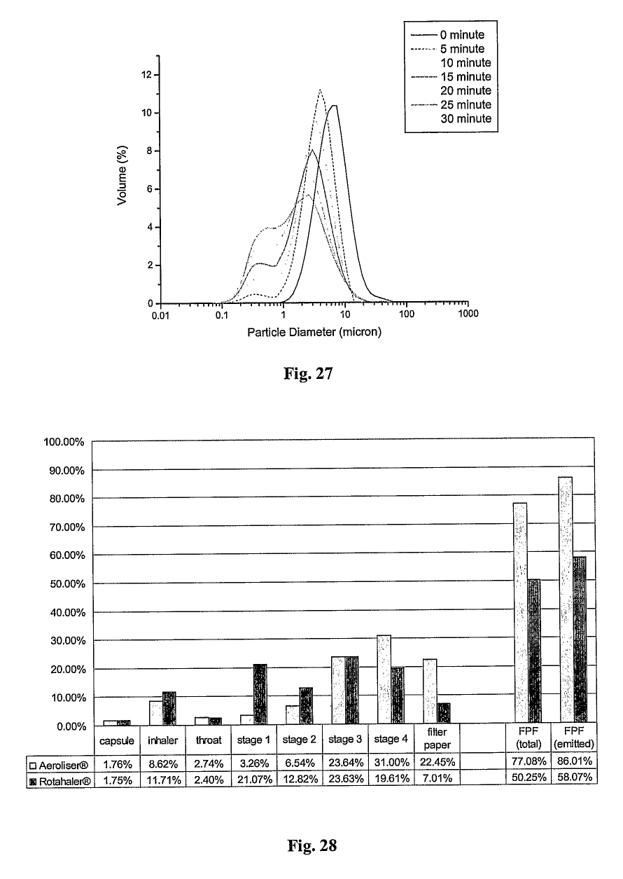

The results shown in FIG. 25 show that, when blended with the lactose, the performance of the blend was not as good as the pure powder. It is known that the function of lactose as a carrier is to improve the inhalation performance. However in the present experiments both FPF values were reduced. The fine salbutamol sulfate may absorb onto the surface of the large lactose particles and may become trapped, such that it is delivered along with the lactose particles mainly to the throat and stage 1 due to the size of lactose particles.

### Reaction Conditions of RPB Synthesis

On account of the differences between beaker synthesis and RPB synthesis, only two of the five reaction parameters could be used unchanged: concentration of Salbutamol base/IPA solution and concentration of sulfuric acid. The stirring speed, as described earlier, translated to the maximum speed that RPB can reach. Reaction temperature and time were investigated separately.

### The Effect of Reaction Temperature

Iced water was used to cool the system, and hot water to heat (Table 9).

TABLE 9
D(v, 0.5) under different reaction temperature

| | Media | Suspension | D(v, 0.5) |
|---|---|---|---|
| | temperature (° C.) | temperature (° C.) | (μm) |
| Cooling | 7 | 17 | 0.84 |
| Room | 20 | 25 | 1.62 |
| temperature | | | |
| Heating | 40 | 30 | 3.40 |

FIG. 26 and Table 9 illustrate that at lower temperature it is possible to achieve finer particles, as for beaker synthesis.

### The Effect of Reactive Time

TABLE 10
Comparison with the Salbutamol sulfate spray dried powder synthesis in RPB

| | | | | D(V, 0.5) | D(V, 0.5) | | |
|---|---|---|---|---|---|---|---|
| sample | | | outlet | before spray | after spray | FPF1 | FPF |
| name | Temp. | time | temp. | drying | drying | (total) | (emitted) |
| 7-Sep. | 25° C. | 10 min | 98° C. | 1.45 | 5.95 | 56.52% | 64.66% |
| 8-Sep. | 25° C. | 20 min | 80° C. | 0.98 | 2.10 | 77.01% | 89.13% |
| 13-Sep. | 25° C. | 30 min | 89° C. | 0.93 | 1.90 | 78.39% | 87.91% |

From table 10, it can be seen that particle size decreased and FPF increased with an increase in reaction time, however there was not a large difference between 20 minutes and 30 minutes.

It was shown that at different reaction times the sample had a different particle size distribution. The main peak of the curve was shifted and decreased in area, and a small peak appeared from about 5 minutes, which progressively increased. D(v,0.5) decreased over time. In conclusion, finer particles could be obtained by employing a longer reaction time.

### Effect of Different Inhalation Device

In order to examine the performance of Salbutamol sulfate spray dry powder synthesed using RPB, Rotahaler® was used to compare with Aeroliser® as before. As observed earlier, the performance of Rotahaler® was worse than Aeroliser®. Approximate 30% reductions in both FPF(total) and FPF(emitted) were observed. However, these values are still above 50%, which is regarded as moderate performance. Thus it has been shown that the Salbutamol sulfate spray dried powder synthesised using RPB has approximately equivalent performance to that synthesized in a beaker.

### SEM Photos

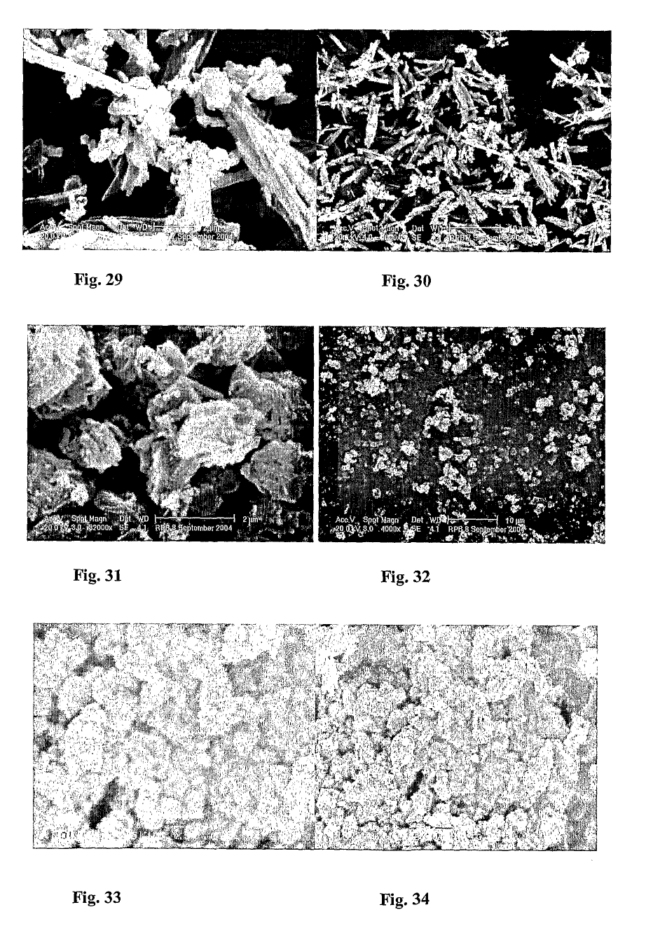

Sep. 7 sample (FIGS. 29 and 30)

| | | | | D(V, 0.5) | D(V, 0.5) | | |
|---|---|---|---|---|---|---|---|
| sample | | | outlet | before spray | after spray | FPF1 | FPF |
| name | Temp. | time | temp. | dry | dry | (total) | (emitted) |
| 7-Sep. | 25° C. | 10 min | 98° C. | 1.45 | 5.95 | 56.52% | 64.66% |

Sep. 8 sample (FIGS. 31 and 32)

| | | | | D(V, 0.5) | D(V, 0.5) | | |
|---|---|---|---|---|---|---|---|
| sample | | | outlet | before spray | after spray | FPF1 | FPF |
| name | Temp. | time | temp. | dry | dry | (total) | (emitted) |
| 8-Sep. | 25° C. | 20 min | 80° C. | 0.98 | 2.10 | 77.01% | 89.13% |

Sep. 13 sample (FIGS. 33 and 34)

| | | | | D(V, 0.5) | D(V, 0.5) | | |
|---|---|---|---|---|---|---|---|
| sample | | | outlet | before spray | after spray | FPF1 | FPF |
| name | Temp. | time | temp. | dry | dry | (total) | (emitted) |
| 13-Sep. | 25° C. | 30 min | 89° C. | 0.93 | 1.90 | 78.39% | 87.91% |

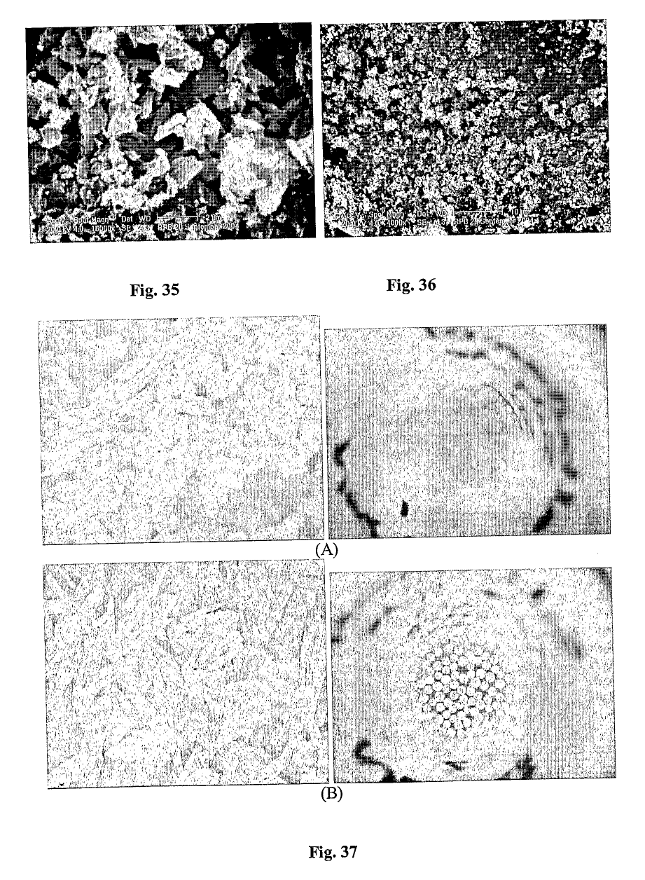

Sep. 30 sample (FIGS. 35 and 36)

| | | | | D(V, 0.5) | D(V, 0.5) | | |
|---|---|---|---|---|---|---|---|
| sample | | | outlet | before spray | after spray | FPF1 | FPF |
| name | Temp. | time | temp. | dry | dry | (total) | (emitted) |
| 30-Sep. | 17° C. | 30 min | 85° C. | 0.84 | 1.98 | 63.83% | 78.87% |

The morphology of the samples from Sep. 8, 13 and 30 all appear similar under the scanning electron microscope, where individual bar shaped particles form spherical agglomerates. The length of the individual particle was under 2 μm, the width and thickness were around 100˜200 nm. The agglomerates of the Sep. 7 sample were larger than those of the other samples. The individual particles of this sample were more like a needle shape of about 10 μm in length and 1 μm in the other two dimensions. This accords with the fact that the FPFs of Sep. 7 sample were the lowest of the four samples, since smaller particle size correlates with higher FPF values.

### Non-solvent Method

The spray drying parameters were the same as those used previously. An interesting phenomenon was observed regarding the dry powder in the spray dryer collected vial. The powder separated into two portions, one spherical and the other loose, which stuck on the side wall of the vial. On measuring the mass and volume of these two portions of powder, it was found that the density of the spherical powder was the twice that of the loose powder. The particle size distribution was measured using Malvern Mastersizer.

It was apparent that the different shapes of dry powder had different morphology under SEM. As discussed above, short bar shape particles provide better performance than long needle shape ones. It was therefore postulated that these two differently shaped powder portions might provide different dispersion results, and that the loose one would be better than the spherical one.

### Dispersion Results

Two separate capsules containing the loose powder stuck and the spherical powder from the spray dryer were prepared for dispersion experiments. The results are shown in FIG. 37.

These results matched the postulated dispersion resulted discussed above. Due to the different sizes of the particles, different FPF values and powder distributions in MSLI were obtained. Because of the relatively large particle sizes, these two samples both exhibited low FPF values. The spherical powder had approximately the same value as the commercial product.

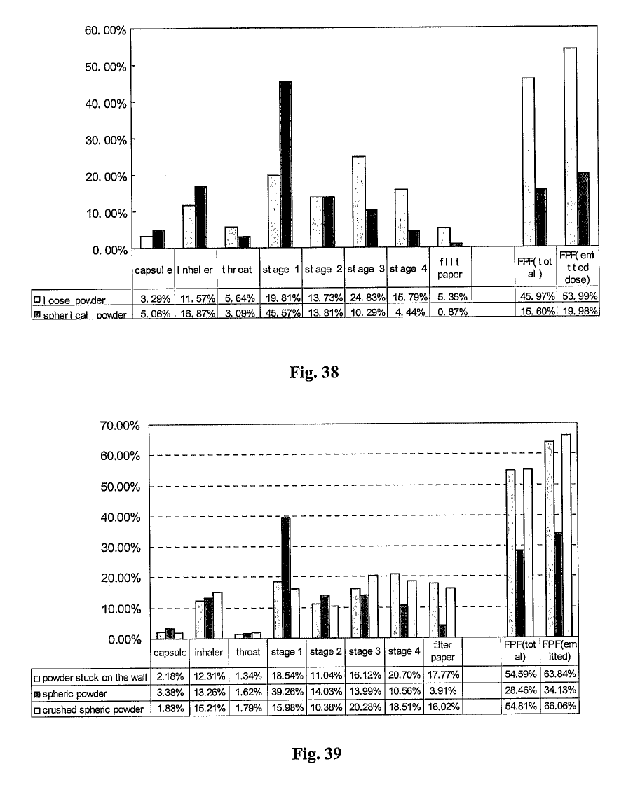

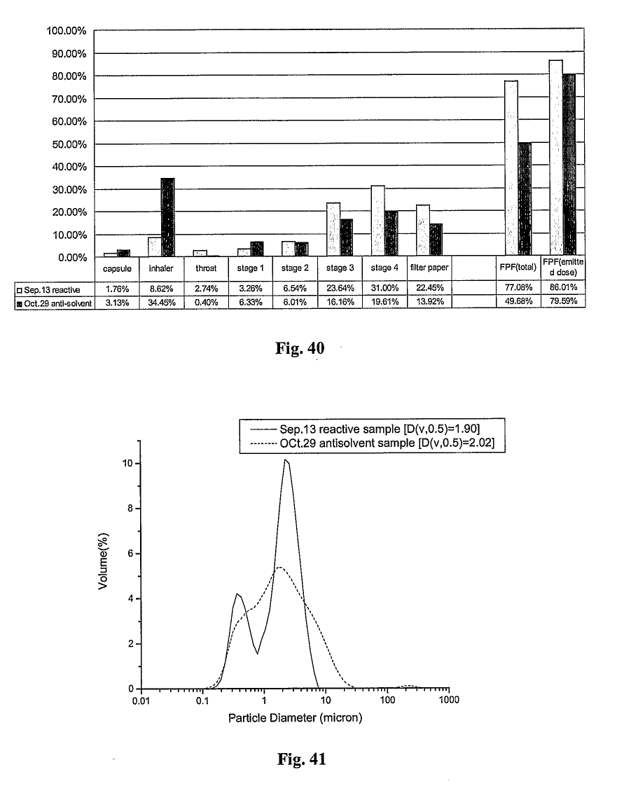

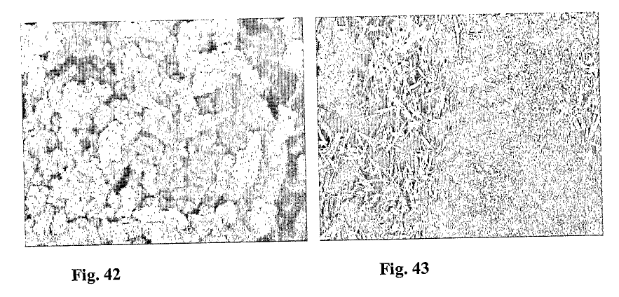

A set of experiments was conducted to confirm the above results using the sample prepared (synthesized and spray-dried) on the 23rd of November 2004. Three separate capsules were made containing loose powder, spherical powder, and crushed spherical powder. These were dispersed individually, and the results are shown in FIG. 38.

The loose powder and crushed spherical powder exhibited the same performance, however the values for the spherical powder were quite low. This suggests that there was not a great difference between the unagglomerated forms of these two powders. It appears that the reason that the spherical powder left a substantial amount of material in stage one is that the agglomerates were too tight to be broken up by the air at a speed of 60 L/min, and although the holes in the capsule made by the Aeroliser® inhaler help break the agglomerates enough to escape the capsule, it is not enough to perform as well as the others. Those agglomerates which did not break enough remained trapped within the capsule, more than if it was just loose or crushed powder.

### Comparison of Powder Generated by Two Methods

### Parameters

Table 11 shows the parameters used in precipitating and spray drying processes.

TABLE 11
Parameters

Sep. 13 reactive method sample

| Precipitating | Concentration and | 10 | mg (Salbutamol |
|---|---|---|---|
| | volume of solution | | base)/ml(IPA); |
| | | 100 | ml |
| | Concentration | 2 | mol/L; |
| | and volume of | 1 | ml |
| | sulfuric acid | | |
| | Temperature | 25° C. | |
| | Time | 30 | minutes |
| | Stir speed | 50 | Hz |
| Spray drying | Inlet temperature | 150° C. | |
| | Outlet temperature | 89° C. | |
| | Aspirator | 100% | |
| | Feed flow rate | 5 | ml/min |
| | Air flow rate | 800 | L/hr |

| | Oct. 29 non-solvent method sample | | |
|---|---|---|---|
| Precipitating | Concentration and | 1 | g (Salbutamol sulfate)/ |
| | volume of solution | 4 | ml deionised water |
| | Volume of non- | 80 | ml (IPA) + |
| | solvent | 20 | ml (acetone) |
| | Temperature | 25° C. | |
| | Time | 30 | minutes |
| | Stir speed | 50 | Hz |
| Spray drying | Inlet temperature | 150° C. | |
| | Outlet temperature | 85° C. | |
| | Aspirator | 100% | |
| | Feed flow rate | 5 | ml/min |
| | Air flow rate | 800 | L/hr |

### Dispersion Results

As shown in Table 11, the parameters used in the two methods to generate the Salbutamol sulfate dry powder were very similar. The dry powder made using the non-solvent method showed significantly poorer performance compared to the sample produced using the reactive method. The majority of the powder (non-solvent method) was deposited in the inhaler instead of in stages 3, 4, and filter paper. It appears likely that there were some particles larger than 15 μm in the Oct. 29 sample. Relative to the FPF(total), there was only about 6% difference between FPF(emitted) of the two samples. This indicates that the powder emitted into the MSLI did have good performance, i.e. there were still fine particles in the dry powder, and/or the air flow is strong enough to break and disperse the agglomerates emitted from the inhaler.

### Particle Size Distribution

Although, as indicated by the results above, the two samples had approximately the same volume medium diameter, however the shapes of distribution curves (FIG. 41) of each were very different. The Sep. 13 sample showed two peaks, and the peak width of each was narrow. However, the Oct. 29 sample had a comparatively broad distribution with a single peak. As the figure showed, all particles of Sep. 13 sample were below 10 μm, but the largest particle size of the Oct. 29 sample was about 30 μm, which is 3 times larger than the maximum size in the Sep. 13 sample. This provides an explanation for the low performance of Oct. 29 sample.

### SEM Photos

The SEM photos explained the dispersion results further. As discussed above, particle size and particle shape are expected to influence FPF values. For the Oct. 29 sample, the loose powder and spherical powder were separated before preparing the SEM sample. Consequently, in the electron micrograph of FIG. 43, two different shapes and sizes of particles may be observed. The small particles were similar to the sample prepared by the reaction sample, and were deposited in stage 3, 4 and filter paper in MSLI. The large particles were those stuck on the side of inhaler due to of the large surface area.

1. A process for preparation of particles of an inhalable drug comprising combining a first liquid and a second liquid in a region of high shear, whereby the first liquid and the second liquid interact to form the particles of the drug, and whereby where one of the liquids comprises a precursor, and the other of the first and second liquids comprises a reagent which reacts with the precursor under high shear conditions to form particles of the drug.

2. The process of claim 1 wherein the particles are of a size suitable for administration by inhalation.

3. The process of claim 2 wherein the particles are less than about 10 microns in diameter.

4. The process of claim 3 wherein the particles are between about 0.5 and about 10 microns in diameter.

5. The process of claim 1 wherein the reagent is capable of reacting with the precursor through an acid-base reaction to form the drug.

6. The process of claim 1 wherein the ratio of the precursor to the reagent is between about 3:1 and about 1:3 on a mole basis.

7. The process of claim 1 wherein drug is a salt, the precursor is a free base of the drug and the reagent is an acid.

8. The process of claim 1 wherein the drug is salbutamol sulfate, the precursor is salbutamol and the reagent is sulfuric acid.

9. The process of claim 1 wherein the high sheer is provided by a shear device rotating in a mixing zone.

10. The process of claim 9 wherein the shear device is rotating in the mixing zone at between about 1,000 and about 10,000 rpm.

11. An inhalable drug made by the process of claim 1

12. The inhalable drug of claim 11 having a narrow particle size distribution.

13. The inhalable drug of claim 11 wherein said drug is selected from the group consisting of an inhalable steroid, an inhalable cyclosporine, an inhalable anti-asthma drug, an inhalable bronchodilator, an inhalable antibiotic and an inhalable β-agonist.

14. A method for treating a condition in a patient comprising providing to the patient an inhalable drug of claim 11.

15. The method of claim 14 wherein the drug does not have a liquid carrier.

16. The method of claim 14 wherein the condition is selected from the group consisting of asthma, cancer and an infection.

17. An inhaler for treating a condition in a patient with particles of an inhalable drug according to claim 11 therein, said drug being suitable, or indicated, for the condition, and said particles having a particle diameter between about 0.5 and about 10 microns.

18. The inhaler of claim 17 wherein the condition is selected from the group consisting of asthma, cancer and an infection.

19. (canceled)
